SEQUENCE LISTING (31 sequences)

SEQ ID NO: 1 - protein, 118 aa, Artificial Sequence
  HCVR
EVQLQQSGPELLKPGASVKMSCKASGYIFTDYFMNWVKQSHGKSLEWIGRVNPKNGDITYNQKFRGKATLTVDKSLTTAYMQLNSLTSEDSAVYFCAYDGYYDGMDFWGQGTSVTVSS

SEQ ID NO: 2 - protein, 108 aa, Artificial Sequence
  LCVR
ENVLTQSPAIMSASPGEKVTMTCRANSSVSSNFLHWYQQKSGASPKLWISSTSNLAFGVPPRFSGSGSGTSYSLTISSVEAEDAATYYCHQYSGHPFTFGSGTKLEIK

SEQ ID NO: 3 - protein, 121 aa, Artificial Sequence
  HCVR
QVQLQQSGAELVRPGASVKLSCKASGYVFPDYEMHWVKQTPVHGLEWIGAFDPETGDTTYNQNFKGKATLTADKASSTAYMDLRSLTSGDSAVYYCARDYKYLYYYAMDNWGQGTSVTVSS

SEQ ID NO: 4 - protein, 108 aa, Artificial Sequence
  LCVR
QIVLTQSPAIMSASLGERVTMTCTASSSITSSYLHWYQQKPGSSPKLWIYSTTNLASGVPGRFSGSGSGTSFSLTISSMEAEDAATYYCHQYRRSPLTFGAGTKLELK

SEQ ID NO: 5 - protein, 117 aa, Artificial Sequence
  HCVR
QIQLQQSGPELVKPGASVKISCKASGYTFTDYYLNWVKQRPGQGLEWIGLIFPGNDKTEYSEKFKGKATLTVDTSSSTAYIQLSRLSSEDSAIYFCSGGDYGGFVYWGQGTLVTVSS

SEQ ID NO: 6 - protein, 107 aa, Artificial Sequence
  LCVR
DIQMTQSPTSLSASVGETVTITCGASENIYGALNWYQRKQGKSPQLLIYGATNLADGMSSRFSGSGSGRQFSLKINSLHPDDVATYFCQNVLSSPLTFGSGTKLEIK

SEQ ID NO: 7 - protein, 118 aa, Artificial Sequence
  HCVR - VH H41C
EVQLQQSGPELLKPGASVKMSCKASGYIFTDYFMNWVKQSCGKSLEWIGRVNPKNGDITYNQKFRGKATLTVDKSLTTAYMQLNSLTSEDSAVYFCAYDGYYDGMDFWGQGTSVTVSS

SEQ ID NO: 8 - protein, 121 aa, Artificial Sequence
  HCVR - VH P41C
QVQLQQSGAELVRPGASVKLSCKASGYVFPDYEMHWVKQTCVHGLEWIGAFDPETGDTTYNQNFKGKATLTADKASSTAYMDLRSLTSGDSAVYYCARDYKYLYYYAMDNWGQGTSVTVSS

SEQ ID NO: 9 - protein, 117 aa, Artificial Sequence
  HCVR - VH P41C
QIQLQQSGPELVKPGASVKISCKASGYTFTDYYLNWVKQRCGQGLEWIGLIFPGNDKTEYSEKFKGKATLTVDTSSSTAYIQLSRLSSEDSAIYFCSGGDYGGFVYWGQGTLVTVSS

SEQ ID NO: 10 - protein, 118 aa, Artificial Sequence
  humanized HCVR
QVQLVQSGAEVKKPGASVKVSCKASGYIFTDYFMNWVRQACGQGLEWMGRVNPKNGDITYNQKFRGRVSLTKDTSINTAYMELSGLTSDDTAIYYCAYDGYYDGMDFWGQGTSVTVSS

SEQ ID NO: 11 - protein, 108 aa, Artificial Sequence
  humanized LCVR
ELVMTQSPSSLSASVGDRVTITCRANSSVSSNFLHWYQQKPGKAPKLLISSTSNLAFGVPSRFSGTGSGTDFSLTISSLQPEDFAIYYCHQYSGHPFTFGQGTKLEIK

SEQ ID NO: 12 - protein, 121 aa, Artificial Sequence
  humanized HCVR
QVQLVQSGAELRKPGSSVKVSCRASGYVFPDYEMHWVRQACGQGLEWMGAFDPETGDTTYNQNFKGRVTIAADKSTSTAYMELRSLRSEDTAIYYCARDYKYLYYYAMDNWGQGTMVTVSS

SEQ ID NO: 13 - protein, 121 aa, Artificial Sequence
  humanized HCVR
QVQLVQSGAEVKKPGSSVKVSCKASGYVFPDYEMHWVRQACGQGLEWMGAFDPETGDTTYNQNFKGRVTITADKSTSTAYMELSSLRSEDTAVYYCARDYKYLYYYAMDNWGQGTTVTVSS

SEQ ID NO: 14 - protein, 108 aa, Artificial Sequence
  humanized LCVR
DIQMTQSPSSLSASVGDRVTITCTASSSITSSYLHWYQQKPGKAPKLLIYSTTNLASGVPSRFSGSGSGTDFTLTISSLQPEDFATYYCHQYRRSPLTFGGGTKLEIK

SEQ ID NO: 15 - protein, 108 aa, Artificial Sequence
  humanized LCVR
EIVLTQSPSLLSASIGDRVTIPCTASSSITSSYLHWYQQKPGKAPKLLIYSTTNLASGVPSRFSGSGSGTEFTLTISSLQPEDFATYYCHQYRRSPLTFGGGTKLEIK

SEQ ID NO: 16 - protein, 117 aa, Artificial Sequence
  humanized HCVR
QVQLVQSGAEVKKPGASVKVSCKASGYTFTDYYLNWVRQACGQGLEWMGLIFPGNDKTEYSEKFKGRVTLTTDTSISTAYMELSSLTSDDTALYYCSGGDYGGFVYWGQGTLVTVSS

SEQ ID NO: 17 - protein, 117 aa, Artificial Sequence
  humanized HCVR
QVQLQESGAEVKKPGASVKVSCKASGYTFTDYYLNWVRQACGQGLEWMGLIFPGNDKTEYSEKFKGRVTMTRDTSTSTVYMELSSLRSEDTAVYYCSGGDYGGFVYWGQGTLVTVSS

SEQ ID NO: 18 - protein, 107 aa, Artificial Sequence
  humanized LCVR
DIQMTQSPSSLSASVGDRVTITCGASENIYGALNWYQQKPGKAPKLLIYGATNLADGVPSRFSGSGSGTDFTLTISSLQPEDVATYYCQNVLSSPLTFGSGTKLEIK

SEQ ID NO: 19 - protein, 107 aa, Artificial Sequence
  humanized LCVR
DIQLTQSPSSLSASVGDRVTITCGASENIYGALNWYQQKPGKAPKLLIYGATNLADGVPSRFSGSGSGTDFTLTISSLQPEDFATYYCQNVLSSPLTFGPGTKVDIK

SEQ ID NO: 20 - protein, 107 aa, Artificial Sequence
  humanized LCVR
DIQMTQSPSSLSASVGDRVTISCGASENIYGALNWYQQQPGKAPKVLIFGATNLADGVPSRFSGSGSGTDFTLTISSLQPEDFATYYCQNVLSSPLTFGQGTKLEIK

SEQ ID NO: 21 - protein, 21 aa, Artificial Sequence
  HAVT20 leader
MACPGFLWALVISTCLEFSMA

SEQ ID NO: 22 - protein, 330 aa, Artificial Sequence [sequence omitted]

SEQ ID NO: 23 - protein, 107 aa, Artificial Sequence [sequence omitted]

SEQ ID NO: 24 - protein, 1390 aa, Artificial Sequence
  human c-Met
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 25 - protein, 1385 aa, Artificial Sequence
  cynomolgus monkey c-Met
MKAPAVLVPGILVLLFTLVQRSNGECKEALAKSEMNVNMKYQLPNFTAETAIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPNQCPDCVVSALGAKVLSSVKDRFINFFVGNTINSSYFPHHPLHSISVRRLKETKDGFMFLTDQSYIDVLPEFRDSYPIKYIHAFESNNFIYFLTVQRETLNAQTFHTRIIRFCSLNSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRAEFTTALQRVDLFMGQFSEVLLTSISTFVKGDLTIANLGTSEGRFMQVVVSRSGPSTPHVNFLLDSHPVSPEVIVEHPLNQNGYTLVVTGKKITKIPLNGLGCRHFQSCSQCLSAPPFVQCGWCHDKCVRSEECPSGTWTQQICLPAIYKVFPTSAPLEGGTRLTICGWDFGFRRNNKFDLKKTRVLLGNESCTLTLSESTMNTLKCTVGPAMNKHFNMSIIISNGHGTTQYSTFSYVDPIITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLHSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISIALLLLLGLFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCALMSPPLDFLGITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDT

SEQ ID NO: 26 - protein, 5 aa, Artificial Sequence
  HC CDR1
DYYLN

SEQ ID NO: 27 - protein, 17 aa, Artificial Sequence
  HC CDR2
LIFPGNDKTEYSEKFKG

SEQ ID NO: 28 - protein, 8 aa, Artificial Sequence
  HC CDR3
GDYGGFVY

SEQ ID NO: 29 - protein, 11 aa, Artificial Sequence
  LC CDR1
GASENIYGALN

SEQ ID NO: 30 - protein, 7 aa, Artificial Sequence
  LC CDR2
GATNLAD

SEQ ID NO: 31 - protein, 9 aa, Artificial Sequence
  LC CDR3
QNVLSSPLT

(19) United States
(12) Patent Application Publication
(10) Pub. No.: US 2024/0376214 A1
(43) Pub. Date: Nov. 14, 2024

(54) ANTI-C-MET ANTIBODIES AND ANTIBODY-DRUG CONJUGATES

(71) Applicant: BYONDIS B.V., Nijmegen (NL)

(72) Inventors: Marion BLOMENROHR, Nijmegen (NL); Miranda Maria Cornelia VAN DER LEE, Nijmegen (NL); Patrick Gerhard GROOTHUIS, Nijmegen (NL)

(21) Appl. No.: 18/554,339

(22) PCT Filed: Apr. 6, 2022
(86) PCT No.: PCT/EP2022/059059

§ 371 Date: Oct. 6, 2023

(30) Foreign Application Priority Data
Apr. 8, 2021 (EP) 21167423.9

(51) Int. Cl. C07K 16/28; A61K 39/00; A61K 47/68; A61P 35/00

(52) U.S. Cl. CPC C07K 16/2863; A61K 47/6803; A61K 47/6849; A61K 47/6889; A61P 35/00; A61K 2039/505; C07K 2317/24

(57) ABSTRACT

The present invention relates to antibodies or antigen-binding fragments thereof that specifically bind to the mesenchymal-epithelial transition factor (c-Met). The invention additionally relates to antibody drug conjugates (ADCs) comprising these anti-c-Met antibodies or antigen-binding fragments, pharmaceutical compositions comprising the antibodies, antigen-binding fragments or ADCs, and the use thereof in the treatment of cancer.

### FIELD OF THE INVENTION

The present invention relates to antibodies or antigen-binding fragments thereof that specifically bind to the mesenchymal-epithelial transition factor (c-Met). The invention additionally relates to antibody drug conjugates (ADCs) comprising these anti-c-Met antibodies or antigen-binding fragments, pharmaceutical compositions comprising the antibodies, antigen-binding fragments or ADCs, and the use thereof in the treatment of cancer.

### BACKGROUND OF THE PRESENT INVENTION

The hepatocyte growth factor receptor or mesenchymal-epithelial transition factor (HGFR, c-Met) is a receptor tyrosine kinase encoded by the MET oncogene and expressed on the surface of various epithelial cells. The ligand for c-Met is hepatocyte growth factor (HGF), also known as scatter factor (SF), a large molecular weight polypeptide known for its angiogenic and mitogenic properties.

The extracellular portion of mature human c-Met is composed of three domain types: 1) a semaphorin (SEMA) domain which is formed by the folding of the 500 N-terminal residues and encompasses the whole alpha-subunit and part of the beta-subunit; 2) a PSI domain (found in plexins, semaphorins, and integrins) of approximately 50 residues and including four disulphide bonds; and 3) four immunoglobulin-plexin-transcription (IPT) domains which connect the PSI domain to the transmembrane helix. Intracellularly, c-Met contains a tyrosine kinase catalytic domain flanked by distinctive juxtamembrane and carboxy-terminal sequences. Within the folded structure of the c-Met protein, the SEMA domain forms a beta propeller with 7 blades.

c-Met is expressed in many different normal tissues (Human Protein Atlas, www.proteinatlas.org, and internal findings). It is most prominently observed in gastrointestinal tissues (i.e., stomach, gall bladder, duodenum, small intestine, colon, rectum), female reproductive tissues (i.e., endometrium, cervix, vagina, placenta), urogenital tissues (i.e., bladder, ureter, kidney), and to some extent also in inter alia thyroid gland, skin, lung, liver, breast, and esophagus. In addition, prominent c-Met expression is present in the eye (i.e., corneal and lens epithelium, limbal region and conjunctiva), as well as in the eye lids (i.e., in lacrimal glands, Meibomian glands, sebaceous glands, hair sheaths).

Binding of HGF to c-Met leads to receptor dimerization, heteromerization or multimerization, phosphorylation of multiple tyrosine residues in the intracellular region, and activation of a wide range of different cellular signalling pathways, including those involved in proliferation, motility, migration, and invasion. Although c-Met is important in the control of tissue homeostasis under normal physiological conditions, it is also evidently involved in the development and progression of malignancies via (exon 14) mutation, gene amplification, or protein overexpression. c-Met-related mechanisms also appear to be involved in resistance to (chemo)therapies, for example therapies aimed at other regulators of cell proliferation such as Epidermal Growth Factor Receptor (EGFR), Transforming Growth Factor-β (TGF-β), and Human Epidermal growth factor Receptor 3 (HER3).

The human c-Met signalling pathway is one of the most frequently dysregulated pathways in human cancers. It is implicated in many types of solid tumors and high c-Met expression is generally associated with poor prognosis. For this reason, the c-Met, HGF/SF signalling pathway has become a target for cancer therapy.

A large variety of c-Met inhibitors ranging from small molecules to antibodies have been in clinical development, but the clinical outcomes have been disappointing, as positive responses were only seen in patients with tumors that are dependent on c-Met signalling, as for instance tumors with MET amplifications or exon 14 mutations, and in some cases therapies have even aggravated the patient's condition.

Approved small molecule c-Met inhibitors include tyrosine kinase inhibitors (TKIs) crizotinib (Xalkori®, Pfizer), used in the treatment of anaplastic lymphoma kinase (ALK)-positive or ROS1 tyrosine kinase-positive non-small cell lung cancer (NSCLC), and cabozantinib (Cometriq®, Ipsen Pharma; Cabometyx®, Ipsen Pharma), which targets inter alia c-Met and VEGFR2 and is used in the treatment of medullary thyroid carcinoma and renal cell carcinoma (RCC).

In the context of cancer therapy, any agonistic effect on c-Met should be avoided. Therefore, a suitable therapeutic antibody interacts with c-Met in such a way that c-Met dimerization and consequent activation (agonistic effect) is avoided, while internalization and degradation is induced. Although also agonistic antibodies, for the use in regenerative medicine, may be created by design (e.g., WO 2018/001909), these antibodies are often the unwanted byproduct of a search for suitable therapeutic antibodies with an antagonistic (i.e., inhibitory) effect on c-Met signalling, to be used in cancer treatment.

Designing anti-c-Met antibodies for use as cancer therapeutics, thus involves a delicate balance between both favorable binding characteristics, inhibition of c-Met signalling, and acceptable pharmacokinetic and pharmacodynamic properties.

The search for an antibody that has an antagonistic, but no agonistic effect is a complex task. Some antibodies may even flip from antagonistic to agonistic, for example, as the result of a humanization process on the basis of mouse monoclonal antibodies.

Several antibodies targeting c-Met for use in cancer therapy have been developed and clinical trials were initiated. Such c-Met specific antibodies include conventional anti-c-Met antibodies, as well as bispecific antibodies, targeting both c-Met and other signalling proteins, such as EGFR.

Antagonistic antibodies against c-Met that were taken into clinical development are, for example, onartuzumab (Genentech, WO 2006/015371), ARGX-111 (Argenx, WO 2012/059561), emibetuzumab (LY2875358; Eli Lilly, WO 2010/059654), SAIT-301 (Samsung, US 2014-0154251), telisotuzumab (antibody ABT-700; Abbott/Abbvie, Wang et al., BMC Cancer 2016, 16, 105-118; WO 2017/201204), and Sym015 (a mixture of two anti-c-Met monoclonal antibodies which bind to non-overlapping epitopes in the c-Met ECD; Symphogen, WO 2016/042412).

Onartuzumab was the first developed anti-c-Met antibody, a humanized, monovalent, antagonistic anti-c-Met antibody derived from the c-Met agonistic antibody 5D5 (Spigel et al., J. Clin. Oncol. 2013, 31, 4105-4114; Xiang et al., Clin. Cancer Res. 2013, 19, 5068-5078). Despite promising experimental results, the development of onartuzumab was terminated due to a lack of clinically meaningful efficacy in a late stage clinical trial (Spigel et al., J. Clin. Oncol. 2014, 32, abstract 8000; NCT01456325). Humanized IgG4 emibetuzumab (LY2875358) was the subject of a Phase 2 clinical trial (NCT01900652) in patients with stage IV EGFR-mutant NSCLC. In this study, no significant difference in median Progression Free Survival (PFS) was observed in the intent-to-treat population (9.3 months with emibetuzumab plus erlotinib versus 9.5 months with erlotinib monotherapy) (Scagliotti et al., J. Thorac. Oncol. 2020, 15, 80-90). Human IgG1 ARGX-111 (NCT02055066), humanized IgG1 ABT-700 (telisotuzumab; NCT01472016) and humanized monoclonal IgG1 antibody mixture Sym015 (NCT02648724) have been evaluated in clinical Phase 1. Currently, no anti-c-Met antibodies have been approved for therapeutic use.

ADCs are an interesting alternative, in part because they are also capable of killing cells that are not dependent on the c-Met signalling pathway. Whereas the efficacy of c-Met-targeting TKIs or monoclonal antibodies is largely dependent on c-Met-driven tumors/MET-amplified tumors, the efficacy of an ADC comprising an antibody to c-Met largely depends on extracellular c-Met-expression and internalization, as well as sensitivity to the cytotoxic agent coupled to the antibody in the ADC.

Anti-c-Met antibodies deemed suitable for ADCs that will be used in the treatment of cancer should thus bind to c-Met with high affinity, and have acceptable pharmacokinetic and pharmacodynamic properties, whereas they should not have an agonistic effect. Additionally, they should induce c-Met internalization.

As described hereinabove, the search for an antibody that has an antagonistic, but no agonistic effect already was a complex task, but in the case of antibodies suitable for ADCs it is even more so since epitopes in the extracellular domain (ECD) of c-Met that trigger internalization, but not dimerization, are not fully defined.

For an ADC, the efficacy thus not only depends on the binding characteristics of the antibody (affinity and specificity in terms of antagonistic effect on c-Met), but also on the degree in which the ADC is internalized and subsequently processed by the cell. In the cell the ADC will release the biologically active drug which will then exert its cytotoxic effect. Thus, the sensitivity of tumor cells to the specific cytotoxic payload is important as well.

ADCs based on c-Met specific antibodies include telisotuzumab vedotin (ABBV-399; Abbvie, WO 2017/201204), TR1801-ADC (Tanabe Research, Gymnopoulos et al., Mol. Oncol. 2020, 14, 54-68), SHR-A1403 (Jiangsu HengRui Medicine Co., Yang et al., Acta, Pharmacologica Sinica 2019, 40, 971-979), and hucMET-27-ADCs (Immunogen Inc., WO 2018/129029).

Telisotuzumab vedotin is based on c-Met antibody telisotuzumab (ABT-700) conjugated to monomethyl auristatin E (MMAE) via a cleavable linker. TR1801-ADC is the site-specific conjugate of humanized antibody hD12 and the pyrrolobenzodiazepine (PBD) toxin-linker tesirine. SHR-A1403 is composed of a humanized IgG2 monoclonal antibody against c-Met conjugated to a cytotoxic microtubule inhibitor. The Immunogen ADCs are conjugates of antibody hucMET-27 and indolinobenzodiazepine DNA-alkylating payload DGN549 or DM4.

Several ADCs in the preclinical stage have shown promising results, and some have advanced into clinical trials (NCT02099058, NCT03539536, NCT03859752, NCT03856541), however a survival benefit has yet to be determined.

Despite the fact that development in this area has been ongoing for more than 20 years, no commercial product for use in cancer therapy based on an antibody specific for c-Met is available yet. The development of multiple clinical candidates has been halted, which is another indication that finding an antibody with sufficient specificity, in antagonistic over agonistic effects, on c-Met, as well as an acceptable therapeutic window is far from straightforward.

Nevertheless, in view of the critical role in cancer progression, c-Met remains an attractive target and the need for therapeutic antibodies and ADCs that have the desired selectivity, specificity and efficacy, as well as an acceptable therapeutic window remains.

### BRIEF DESCRIPTION OF THE PRESENT INVENTION

The present invention relates to antibodies or antigen-binding fragments thereof that specifically bind to c-Met, as well as to antibody-drug conjugates (ADCs) comprising these anti-c-Met antibodies or antigen-binding fragments.

In a first aspect, the present invention relates to an antibody or an antigen-binding fragment thereof that specifically binds to c-Met, comprising heavy chain (HC) variable region complementarity determining regions (CDRs) HC CDRs 1-3, wherein
the amino acid sequence of HC CDR1 comprises SEQ ID NO:26;
the amino acid sequence of HC CDR2 comprises SEQ ID NO:27; and
the amino acid sequence of HC CDR3 comprises SEQ ID NO:28;
and light chain (LC) variable region complementarity determining regions (CDRs) LC CDRs 1-3, wherein
the amino acid sequence of LC CDR1 comprises SEQ ID NO:29;
the amino acid sequence of LC CDR2 comprises SEQ ID NO:30; and
the amino acid sequence of LC CDR3 comprises SEQ ID NO:31.

In a second aspect, the present invention relates to an ADC comprising the anti-c-Met antibody or antigen-binding fragment conjugated to a cytotoxic drug through a linker.

In a preferred embodiment, the ADC is of formula (III)

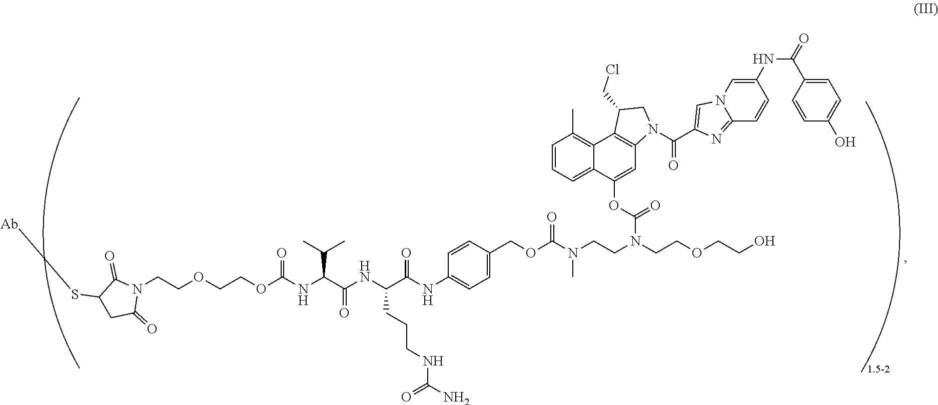

wherein
the HC variable region of Ab is represented by the amino acid sequence of SEQ ID NO:16 and the LC variable region of Ab is represented by the amino acid sequence of SEQ ID NO:20;
Ab is an IgG1 antibody; and wherein
the cytotoxic drug is site-specifically conjugated through the linker to an engineered cysteine on HC position 41 (according to Kabat numbering).

Other aspects of the present invention include a pharmaceutical composition comprising the antibody, antigen-binding fragment or ADC and its use as a medicament, particularly for the treatment of cancer, either in mono- or in combination therapy.

### BRIEF DESCRIPTION OF THE DRAWINGS

FIG. 1. Quantification of internalization kinetics of Alexa Fluor 488 (AF488) labelled mAb3b in c-Met-positive MKN45 cells. (A) Internalization determined on the IncuCyte S3 instrument by monitoring the increase of fluorescent signal internalized in the cytoplasm followed for 24 hours. (B) Percentage internalization measured by flow cytometry.

FIG. 2. Cellular binding of ADC3b and its corresponding unconjugated antibody mAb3b on c-Met positive MKN45 cells.

FIG. 3. In vitro cytotoxicity of ADC3b in human tumor cell lines with varying levels of c-Met expression.

FIG. 4. Bystander cytotoxicity in c-Met-negative Jurkat NucLight Red cells 1:1 co-cultured with c-Met positive MKN45 cells and treated with 1 μg/mL ADC3b or non-binding control ADC.

FIG. 5. In vivo efficacy of chimeric ADCs ADC1, ADC2 and ADC3, and vehicle in a c-Met positive MKN-45 tumor model in female ces1c KO mice.

FIG. 6. In vivo efficacy of humanized ADCs and vehicle in a c-Met positive MKN-45 tumor model in female ces1c KO mice.

FIG. 7. In vivo efficacy of humanized ADCs ADC3a, ADC3b and ADC3c, and vehicle in a c-Met positive MKN-45 tumor model in female ces1c KO mice.

FIG. 8. Efficacy of ADC3b in the non-MET amplified head and neck cancer PDX model HNXF1905 (A) and lung cancer PDX model LXFL1176 (B). The arrow indicates the moment of randomization and dosing of the mice.

### DETAILED DESCRIPTION OF THE PRESENT INVENTION

### Antibodies and Antigen-Binding Fragments Thereof

With the present invention antibodies or antibody binding fragments are provided that have an antagonistic or neutral effect on c-Met without exerting any agonistic effect. A neutral effect means that the antibody binds to c-Met but the binding does not stimulate c-Met signalling.

The present invention relates to an antibody or an antigen-binding fragment thereof that specifically binds to c-Met, defined by its specific complementarity determining regions (CDRs), shows excellent affinity for both human and cynomolgus monkey (cyno) c-Met, as well as a good efficacy, and provides an acceptable therapeutic window.

An antibody or antigen-binding fragment according to the invention comprises a heavy chain (HC) variable region comprising complementarity determining regions (CDRs) HC CDR1-3, wherein the amino acid sequence of HC CDR1 comprises SEQ ID NO:26, the amino acid sequence of HC CDR2 comprises SEQ ID NO:27, and the amino acid sequence of HC CDR3 comprises SEQ ID NO:28.

An antibody or antigen-binding fragment according to the invention further comprises a light chain (LC) variable region comprising complementarity determining regions LC CDR1-3, wherein the amino acid sequence of LC CDR1 comprises SEQ ID NO:29, the amino acid sequence of LC CDR2 comprises SEQ ID NO:30, and the amino acid sequence of LC CDR3 comprises SEQ ID NO:31.

The term “antibody” as used throughout the specification refers to a monoclonal antibody (mAb) comprising two heavy chains and two light chains. Antibodies may be of any isotype such as IgA, IgE, IgG, or IgM antibodies. Preferably, the antibody is an IgG antibody, more preferably an IgG1 or IgG2 antibody. The antibodies may be chimeric, humanized or human. Preferably, the antibodies of the invention are humanized. Even more preferably, the antibody is a humanized or human IgG antibody, more preferably a humanized or human IgG1 antibody, most preferably a humanized IgG1 antibody. The antibody may have κ (kappa) or λ (lambda) light chains, preferably κ (kappa) light chains, i.e., a humanized or human IgG1-κ antibody.

The antibody or antigen-binding fragment thereof, if applicable, may comprise (1) a constant region that is engineered, i.e., one or more mutations may have been introduced to e.g., increase half-life, provide a site of attachment for a linker-drug and/or increase or decrease effector function; or (2) a variable region that is engineered, i.e., one or more mutations may have been introduced to provide a site of attachment for a linker-drug. Antibodies or antigen-binding fragments thereof may be produced recombinantly, synthetically, or by other known suitable methods.

The term “antigen-binding fragment” as used throughout the specification includes a Fab, Fab′, F(ab′)₂, Fv, scFv or reduced IgG (rIgG) fragment, a single chain (sc) antibody, a single domain (sd) antibody, a diabody, or a minibody.

“Humanized” forms of non-human (e.g., rodent) antibodies are antibodies (e.g., non-human-human chimeric antibodies) that contain minimal sequences derived from the non-human antibody. Various methods for humanizing non-human antibodies are known in the art. For example, the antigen-binding complementarity determining regions (CDRs) in the variable regions (VRs) of the heavy chain (HC) and light chain (LC) are derived from antibodies from a non-human species, commonly mouse, rat or rabbit. These non-human CDRs may be combined with human framework regions (FRs, i.e., FR1, FR2, FR3 and FR4) of the variable regions of the HC and LC, in such a way that the functional properties of the antibodies, such as binding affinity and specificity, are at least partially retained. Selected amino acids in the human FRs may be exchanged for the corresponding original non-human species amino acids to further refine antibody performance, such as to improve binding affinity, while retaining low immunogenicity. The thus humanized variable regions are typically combined with human constant regions. Exemplary methods for humanization of non-human antibodies are the methods of Winter and co-workers (Jones et al., Nature 1986, 321, 522-525; Riechmann et al., Nature 1988, 332, 323-327; Verhoeyen et al., Science 1988, 239, 1534-1536). Alternatively, non-human antibodies can be humanized by modifying their amino acid sequence to increase similarity to antibody variants produced naturally in humans. For example, selected amino acids of the original non-human species FRs are exchanged for their corresponding human amino acids to reduce immunogenicity, while retaining the antibody's binding affinity. For further details, see Jones et al., Nature 1986, 321, 522-525; Riechmann et al., Nature 1988, 332, 323-327; and Presta, Curr. Op. Struct. Biol. 1992, 2, 593-596. See also the following review articles and references cited therein: Vaswani and Hamilton, Ann. Allergy, Asthma and Immunol. 1998, 1, 105-115; Harris, Biochem. Soc. Transactions 1995, 23, 1035-1038; and Hurle and Gross, Curr. Op. Biotech. 1994, 5, 428-433.

The CDRs may be determined using the approach of Kabat (in Kabat et al., Sequences of Proteins of Immunological Interest, 5th Ed. Public Health Service, National Institutes of Health, Bethesda, MD, NIH publication no. 91-3242, pp. 662, 680, 689 (1991)), Chothia (Chothia et al., Nature 1989, 342, 877-883) or IMGT (Lefranc, The Immunologist 1999, 7, 132-136). These methods result in CDRs with slightly varying lengths as is illustrated by the CDRs as underlined in the attached sequences SEQ ID NOs 1-20 which were determined by the method according to IMGT and the CDR sequences as determined by the method of Kabat as illustrated in SEQ ID NOs 26-31.

In one embodiment, the present invention relates to a humanized antibody or an antigen-binding fragment thereof comprising HC CDRs 1-3, wherein
the amino acid sequence of HC CDR1 comprises SEQ ID NO:26;
the amino acid sequence of HC CDR2 comprises SEQ ID NO: 27; and
the amino acid sequence of HC CDR3 comprises SEQ ID NO: 28;
and LC CDRs 1-3, wherein
the amino acid sequence of LC CDR1 comprises SEQ ID NO:29;
the amino acid sequence of LC CDR2 comprises SEQ ID NO: 30; and
the amino acid sequence of LC CDR3 comprises SEQ ID NO:31.

In a preferred embodiment, the antibody or an antigen-binding fragment according to the invention comprises the HC variable region amino acid sequence represented by SEQ ID NO:16 and the LC variable region amino acid sequence represented by SEQ ID NO:20.

In one embodiment, the antibody according to the invention is an intact IgG antibody, more preferably an IgG1 antibody.

In another embodiment, the antibody fragment according to the invention is a Fab, Fab′, or F(ab′)₂ fragment, more preferably a Fab fragment.

Antibodies according to the invention are especially suitable for therapeutic applications due to their high specificity and their excellent affinity for both human and cyno c-Met, whereas they do not exert an agonistic effect either in vitro or in vivo.

### Antibody-Drug Conjugates

The present invention additionally relates to an antibody-drug conjugate (ADC), wherein an antibody or antigen-binding fragment according to the invention is conjugated to a cytotoxic drug, such as a small molecule cytotoxic drug, via a linker. The moiety in which the linker is conjugated to the cytotoxic drug is the linker-drug (moiety).

A linker is preferably a synthetic linker. The structure of a linker is such that the linker can be easily chemically attached to a small molecule cytotoxic drug, and so that the resulting linker-drug can be easily conjugated to a further substance such as for example an antibody or antigen-binding fragment according to the invention to form an antibody-drug conjugate. The choice of linker can influence the stability of such eventual conjugates when in circulation, and it can influence in what manner the small molecule drug compound is released, if it is released. Suitable linkers are for example described in Ducry et al., Bioconjugate Chem. 2010, 21, 5-13, King and Wagner, Bioconjugate Chem. 2014, 25, 825-839, Gordon et al., Bioconjugate Chem. 2015, 26, 2198-2215, Tsuchikama and An (DOI: 10.1007/s13238-016-0323-0), Polakis (DOI: 10.1124/pr.114.009373), Bargh et al. (DOI: 10.1039/c8cs00676 h), WO 2011/133039, WO 2015/177360, and in WO 2018/069375. Linkers may be cleavable or non-cleavable. Cleavable linkers comprise moieties that can be cleaved, e.g., when exposed to lysosomal proteases or to an environment having an acidic pH or a higher reducing potential. Suitable cleavable linkers are known in the art and comprise e.g., a di-, tri- or tetrapeptide, i.e., a peptide composed of two, three or four amino acid residues. Additionally, the cleavable linker may comprise a selfimmolative moiety such as an ω-amino aminocarbonyl cyclization spacer, see Saari et al., J Med. Chem., 1990, 33, 97-101, or a —NH—CH₂—O— moiety. Cleavage of the linker makes the drug moiety in the ADC available to the surrounding environment. Non-cleavable linkers can still effectively release (a derivative of) the drug moiety from the ADC, for example when a conjugated polypeptide is degraded in the lysosome. Non-cleavable linkers include e.g., succinimidyl-4-(N-maleimidomethyl(cyclohexane)-1-carboxylate and maleimidocaproic acid and analogs thereof.

Either a cleavable or a non-cleavable linker may be used in accordance with the present invention. Preferably, the linker has a chemical group which can react with the thiol group of a cysteine residue, typically a maleimide or haloacetyl group. More preferably, the linker is a cleavable linker.

In the context of the invention, the cytotoxic drug that is conjugated to the antibody or antigen-binding fragment according to the invention is suitable for the treatment of cancer. Examples of suitable cytotoxic drugs include, but are not limited to, duocarmycin, calicheamicin, pyrrolobenzodiazepine (PBD) dimer, maytansinoid (e.g., DM1 or DM4) and auristatin (e.g., MMAE or MMAF) derivatives. Preferably, the cytotoxic drug is a duocarmycin derivative.

Duocarmycins, first isolated from a culture broth of Streptomyces species, are members of a family of antitumor antibiotics that include duocarmycin A, duocarmycin SA, and CC-1065. These extremely potent agents allegedly derive their biological activity from an ability to sequence-selectively alkylate DNA at the N3 position of adenine in the minor groove, which initiates a cascade of events that terminates in an apoptotic cell death mechanism.

WO 2011/133039 discloses a series of linker-drugs comprising a duocarmycin derivative of CC-1065. Suitable linker-duocarmycin derivatives to be used in accordance with the present invention are disclosed on pages 182-197. The chemical synthesis of a number of these linker-drugs is described in Examples 1-12 of WO 2011/133039.

In a preferred embodiment, the present invention relates to an ADC, wherein the linker-drug is conjugated to an antibody or antigen-binding fragment according to the invention through a cysteine residue of the antibody or the antigen-binding fragment.

In one embodiment, the present invention relates to an ADC of formula (I)

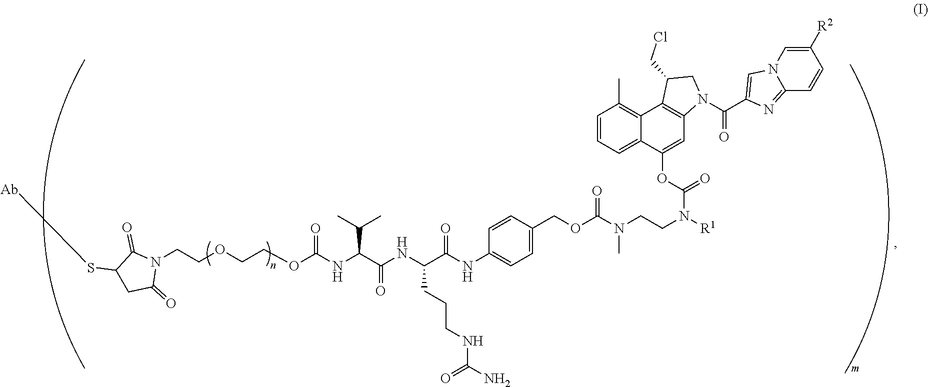

wherein
Ab is an antibody or antigen-binding fragment according to the invention;
n is an integer of from 0-3;
m represents an average DAR of from 1 to 6;
R¹ is selected from the group consisting of

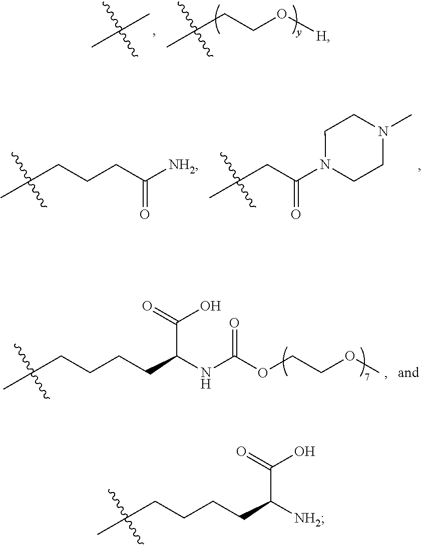

y is an integer of from 1-16; and
R² is selected from the group consisting of

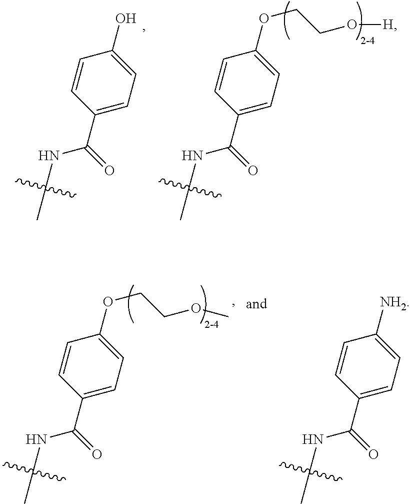

In the structural formulae shown in the present specification, n represents an integer of from 0 to 3, while m represents an average drug-to-antibody ratio (DAR) of from 1 to 6. As is well-known in the art, the DAR and drug load distribution can be determined, for example, by using hydrophobic interaction chromatography (HIC) or reversed phase high-performance liquid chromatography (RP-HPLC). HIC is particularly suitable for determining the average DAR.

In a particular embodiment, the present invention relates to an ADC of formula (I) as disclosed hereinabove, wherein n is 0 or 1, m represents an average DAR of from 1 to 6, preferably of from 1 to 4, more preferably of from 1 to 2, most preferably of from 1.5 to 2, R¹ is selected from the group consisting of

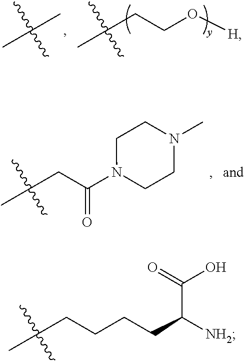

y is an integer of from 1-16, preferably of from 1-4; and
R² is

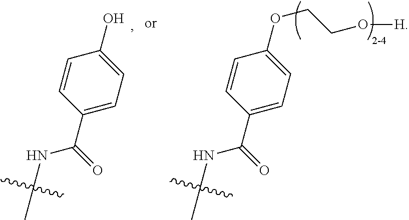

In a specific embodiment, the present invention relates to an ADC of formula (I) as disclosed hereinabove, wherein n is 0 or 1, m represents an average DAR of from 1.5 to 2, R¹ is

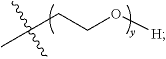

y is of from 1-4; and
R² is

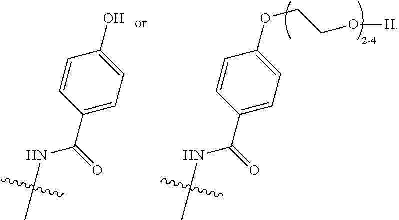

In a preferred embodiment, the ADC is a compound of formula (II)

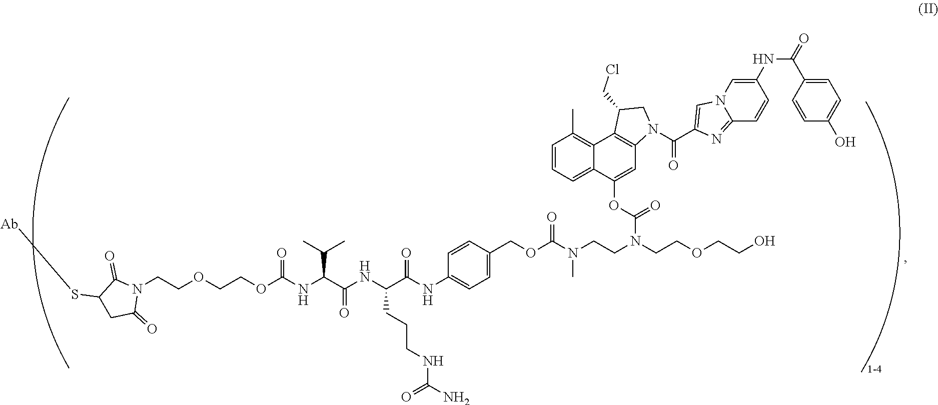

wherein Ab is an antibody or antigen-binding fragment according to the invention and 1-4 represent the average DAR for the compound.

In a particularly preferred embodiment, the ADC is a compound of formula (III)

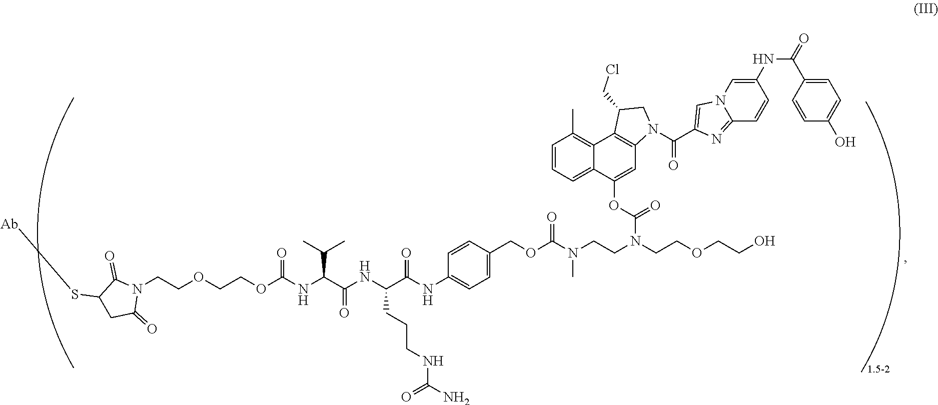

wherein Ab is an antibody or antigen-binding fragment according to the invention 1.5-2 represent the average DAR for the compound.

ADCs of the present invention may be wild-type or site-specific, and can be produced by any method known in the art as exemplified below.

Wild-type ADCs may be produced by conjugating a linker-drug to the antibody or antigen-binding fragment thereof through e.g., the lysine ε-amino groups of the antibody, preferably using a linker-drug comprising an amine-reactive group such as an activated ester; contacting of the activated ester with the antibody or antigen-binding fragment thereof will yield the ADC. Alternatively, wild-type ADCs can be produced by conjugating the linker-drug through the free thiols of the side chains of cysteines generated through reduction of interchain disulfide bonds, using methods and conditions known in the art, see e.g., Doronina et al., Bioconjugate Chem. 2006, 17, 114-124. The manufacturing process involves partial reduction of the solvent-exposed interchain disulfides followed by modification of the resulting thiols with Michael acceptor-containing linker-drugs such as maleimide-containing linker-drugs, alfa-haloacetic amides or esters. The cysteine attachment strategy results in maximally two linker-drugs per reduced disulfide. Most human IgG molecules have four solvent-exposed disulfide bonds, and so a range of integers of from zero to eight linker-drugs per antibody is possible. The exact number of linker-drugs per antibody is determined by the extent of disulfide reduction and the number of molar equivalents of linker-drug used in the ensuing conjugation reaction. Full reduction of all four disulfide bonds gives a homogeneous construct with eight linker-drugs per antibody, while a partial reduction typically results in a heterogeneous mixture with zero, two, four, six, or eight linker-drugs per antibody.

Site-specific ADCs are preferably produced by conjugating the linker-drug to the antibody or antigen-binding fragment thereof through the side chains of engineered cysteine residues in suitable positions of the mutated antibody or antigen-binding fragment thereof. Engineered cysteines are usually capped by other thiols, such as cysteine or glutathione, to form disulfides. These capped residues need to be uncapped before linker-drug attachment can occur. Linker-drug attachment to the engineered residues is either achieved (1) by reducing both the native interchain and mutant disulfides, then re-oxidizing the native interchain cysteines using a mild oxidant such as CuSO₄ or dehydroascorbic acid, followed by standard conjugation of the uncapped engineered cysteine with a linker-drug, or (2) by using mild reducing agents which reduce mutant disulfides at a higher rate than the interchain disulfide bonds, followed by standard conjugation of the uncapped engineered cysteine with a linker-drug. Under optimal conditions, two linker-drugs per antibody or antigen-binding fragment thereof (i.e., drug-to-antibody ratio, DAR, is 2) will be attached (if one cysteine is engineered into the HC or LC of the mAb or fragment). Suitable methods for site-specifically conjugating linker-drugs can for example be found in WO 2015/177360 which describes the process of reduction and re-oxidation, WO 2017/137628 which describes a method using mild reducing agents and WO 2018/215427 which describes a method for conjugating both the reduced interchain cysteines as well as the uncapped engineered cysteines.

In accordance with the present invention, the term “engineered cysteine” means replacing a non-cysteine amino acid in the heavy chain or light chain of an antibody by a cysteine. As is known by the person skilled in the art, this can be done either at the amino acid level or at the DNA level, e.g. by using site-directed mutagenesis.

In one embodiment, the present invention relates to an ADC, wherein a linker-drug is site-specifically conjugated to an antibody or antigen-binding fragment according to the invention through an engineered cysteine residue introduced in the heavy or light chain variable or constant regions.

In a preferred embodiment, the present invention relates to an ADC, wherein a linker drug is site-specifically conjugated to an antibody or antigen-binding fragment according to the invention through an engineered cysteine at one or more positions of said antibody or antigen-binding fragment selected from HC variable region positions 40, 41 and 89 (according to Kabat numbering) and LC variable region positions 40 and 41 (according to Kabat numbering). Preferably, said engineered cysteine is at HC position 41 or LC position 40 or 41, more preferably at HC position 41.

In a specific embodiment, the HC variable region of Ab is represented by the amino acid sequence of SEQ ID NO:16 and the LC variable region of Ab is represented by the amino acid sequence of SEQ ID NO:20. Preferably, the ADC is an ADC of formula (III). More preferably, the cytotoxic drug is site-specifically conjugated through the linker to Ab through an engineered cysteine on HC position 41 (according to Kabat numbering). Even more preferably, Ab is an IgG1 antibody. Most preferably, Ab is an IgG1 antibody with a κ (kappa) light chain.

The ADC comprising the HC variable region represented by SEQ ID NO:16 and LC variable region represented by SEQ ID NO:20, wherein the vc-seco-DUBA drug is site-specifically conjugated through an engineered cysteine on HC position 41 showed a remarkably favorable toxicity profile in cynomolgus monkey, given c-Met's expression in many different normal tissues. Despite this extensive expression, the tolerability of this ADC is surprisingly high. The highest non-severely toxic dose (HNSTD) for the ADC is estimated to be 15 mg/kg/Q3 weeks.

### Pharmaceutical Compositions

The present invention further relates to a pharmaceutical composition comprising an anti-c-Met antibody or antigen-binding fragment thereof, or an anti-c-Met ADC as described hereinabove and one or more pharmaceutically acceptable excipients. Typical pharmaceutical formulations of therapeutic proteins such as mAbs, fragments and (monoclonal) ADCs take the form of lyophilized cakes (lyophilized powders), which require (aqueous) dissolution (i.e., reconstitution) before intravenous infusion, or frozen (aqueous) solutions, which require thawing before use.

Typically, the pharmaceutical composition is provided in the form of a lyophilized cake. Suitable pharmaceutically acceptable excipients for inclusion into the pharmaceutical composition (before freeze-drying) in accordance with the present invention include buffer solutions (e.g., citrate, histidine or succinate containing salts in water), lyoprotectants (e.g., sucrose, trehalose), tonicity modifiers (e.g., sodium chloride), surfactants (e.g., polysorbate), and bulking agents (e.g., mannitol, glycine). Excipients used for freeze-dried protein formulations are selected for their ability to prevent protein denaturation during the freeze-drying process as well as during storage. As an example, the sterile, lyophilized powder single-use formulation of Kadcyla™ (Roche) contains—upon reconstitution with Bacteriostatic or Sterile Water for Injection (BWFI or SWFI)—20 mg/mL ado-trastuzumab emtansine, 0.02% w/v polysorbate 20, 10 mM sodium succinate, and 6% w/v sucrose with a pH of 5.0.

### Medical Uses

In a further aspect, the invention provides an anti-c-Met antibody or antigen-binding fragment thereof, an ADC, or a pharmaceutical composition as described hereinabove for use as a medicament, preferably for use in the treatment of cancer.

A cancer in the context of the present invention, preferably is a tumor expressing c-Met. Such tumor may be a c-Met positive solid tumor or a MET-driven hematological malignancy. Examples of solid tumors or hematological malignancies that may be treated according to the invention as defined above may include, but are not limited to, breast cancer; brain cancer (e.g., glioblastoma multiforme (GBM) or medulloblastoma); head and neck cancer; thyroid cancer; salivary gland cancer (e.g., parotid gland cancer); adrenal cancer (e.g., neuroblastoma, paraganglioma, or pheochromocytoma); bone cancer (e.g., osteosarcoma); soft tissue sarcoma (STS); ocular cancer (e.g., uveal melanoma); esophageal cancer; gastric cancer (GC); small intestine cancer; colorectal cancer (CRC); urothelial cell cancer (e.g., bladder, penile, ureter, or renal cancer); ovarian cancer; uterine cancer (e.g., endometrial cancer); vaginal, vulvar and cervical cancer; lung cancer (especially non-small cell lung cancer (NSCLC) and small-cell lung cancer (SCLC)); melanoma; mesothelioma (especially malignant pleural and abdominal mesothelioma); liver cancer (e.g., hepatocellular carcinoma (HCC)); pancreatic cancer; skin cancer (e.g., basalioma, squamous cell carcinoma, or dermatofibrosarcoma protuberans); testicular cancer; prostate cancer; germ cell cancer; cancer of unknown primary (CUP); and lymphoid (e.g., mature T and NK neoplasms) or myeloid malignancies (multiple myeloma).

In one embodiment, the present invention relates to an anti-c-Met antibody or antigen-binding fragment thereof, an ADC or a pharmaceutical composition as described hereinabove for use in the treatment of a c-Met positive human solid tumor or a MET-driven hematological malignancy, preferably a c-Met positive human solid tumor.

In a preferred embodiment, the present invention relates to an anti-c-Met antibody or antigen-binding fragment thereof, an ADC or a pharmaceutical composition as described hereinabove, particularly an ADC comprising a duocarmycin derivative linker-drug, for use in the treatment of a c-Met positive human solid tumor selected from the group consisting of breast cancer; brain cancer; head and neck cancer; thyroid cancer; salivary gland cancer; soft tissue sarcoma (STS); ocular cancer; esophageal cancer; gastric cancer (GC); small intestine cancer; colorectal cancer (CRC); urothelial cell cancer (UCC); ovarian cancer; uterine cancer; endometrial cancer; cervical cancer; lung cancer (especially non-small cell lung cancer (NSCLC) and small-cell lung cancer (SCLC)); melanoma; liver cancer; pancreatic cancer; non-melanoma skin cancer; prostate cancer; germ cell cancer; and cancer of unknown primary (CUP).

In a more preferred embodiment, the present invention relates to an anti-c-Met antibody or antigen-binding fragment thereof, an ADC or a pharmaceutical composition as described hereinabove, particularly an ADC comprising a duocarmycin derivative linker-drug, for use in the treatment of a c-Met positive human solid tumor selected from the group consisting of breast cancer; glioblastoma multiforme (GBM); medulloblastoma; head and neck cancer; papillary thyroid cancer; salivary gland cancer; soft tissue sarcoma (STS); uveal melanoma; esophageal cancer; gastric cancer (GC); small intestine cancer; colorectal cancer (CRC); urothelial cell cancer (UCC); bladder cancer; urinary tract cancer; penile cancer; papillary renal cell cancer (PRCC); clear cell renal cell cancer (CCRCC); non-clear cell renal cell cancer; nephroblastoma; ovarian cancer; uterine cancer; endometrial cancer; cervical cancer; non-small cell lung cancer (NSCLC); small-cell lung cancer (SCLC); melanoma; hepatocellular carcinoma (HCC); pancreatic cancer; non-melanoma skin cancer; prostate cancer; germ cell cancer; and cancer of unknown primary (CUP).

In a further preferred embodiment, the present invention relates to an anti-c-Met antibody or antigen-binding fragment thereof, an ADC or a pharmaceutical composition as described hereinabove, particularly an ADC comprising a duocarmycin derivative linker-drug, for use in the treatment of a MET-driven human hematological malignancy, wherein the MET-driven hematological malignancy is a lymphoid or myeloid malignancy, more preferably a mature T and NK neoplasm or multiple myeloma.

The anti-c-Met antibody or antigen-binding fragment thereof, ADC or pharmaceutical composition as described hereinabove may be for the use in the manufacture of a medicament as described herein. The anti-c-Met antibody or antigen-binding fragment thereof, ADC or pharmaceutical composition as described hereinabove are preferably for methods of treatment wherein the anti-c-Met antibody or antigen-binding fragment thereof, ADC or pharmaceutical composition are administered to a subject, preferably to a subject in need thereof, in a therapeutically effective amount. Thus, alternatively, or in combination with any of the other embodiments, in an embodiment, the present invention relates to a use of an anti-c-Met antibody or antigen-binding fragment thereof, ADC or pharmaceutical composition as described hereinabove for the manufacture of a medicament for the treatment of cancer. For illustrative, non-limitative, cancers to be treated according to the invention: see hereinabove.

Alternatively, or in combination with any of the other embodiments, in an embodiment, the present invention relates to a method for treating cancer, which method comprises administering to a subject in need of said treatment a therapeutically effective amount of an anti-c-Met antibody or antigen-binding fragment thereof, ADC or pharmaceutical composition as described hereinabove. For illustrative, non-limitative, cancers to be treated according to the invention: see hereinabove.

The anti-c-Met antibody or antigen-binding fragment thereof, ADC or pharmaceutical composition as described hereinabove are for administration to a subject. The anti-c-Met antibody or antigen-binding fragment thereof, ADC or pharmaceutical composition as described hereinabove can be used in the methods of treatment described hereinabove by administration of an effective amount of the composition to a subject in need thereof. The term “subject” as used herein refers to all animals classified as mammals and includes, but is not restricted to, primates and humans. The subject is preferably a human. The expression “therapeutically effective amount” means an amount sufficient to effect a desired response, or to ameliorate a symptom or sign. A therapeutically effective amount for a particular subject may vary depending on factors such as the condition being treated, the overall health of the subject, the method, route, and dose of administration and the severity of side effects.

The present invention further relates to the use of a sequentially or concomitantly administered combination of an anti-c-Met antibody or antigen-binding fragment thereof, an anti-c-Met ADC or a pharmaceutical composition as described hereinabove with one or more other therapeutic agents.

Suitable chemotherapeutic agents include alkylating agents, such as nitrogen mustards, hydroxyurea, nitrosoureas, tetrazines (e.g., temozolomide) and aziridines (e.g., mitomycin); drugs interfering with the DNA damage response, such as PARP inhibitors, ATR and ATM inhibitors, CHK1 and CHK2 inhibitors, DNA-PK inhibitors, and WEE1 inhibitors; anti-metabolites, such as antifolates (e.g., pemetrexed), fluoropyrimidines (e.g., gemcitabine), deoxynucleoside analogues and thiopurines; anti-microtubule agents, such as vinca alkaloids and taxanes; topoisomerase I and II inhibitors; cytotoxic antibiotics, such as anthracyclines and bleomycins; hypomethylating agents such as decitabine and azacitidine; histone deacetylase inhibitors; all-trans retinoic acid; and arsenic trioxide. Suitable radiation therapeutics include radio-isotopes, such as ¹³¹I metaiodobenzylguanidine (MIBG), ³²P as sodium phosphate, ²²³Ra chloride, ⁸⁹Sr chloride and ¹⁵³Sm diamine tetramethylene phosphonate (EDTMP). Suitable agents to be used as hormonal therapeutics include inhibitors of hormone synthesis, such as aromatase inhibitors and GnRH analogues; hormone receptor antagonists, such as selective estrogen receptor modulators (e.g., tamoxifen and fulvestrant) and antiandrogens, such as bicalutamide, enzalutamide and flutamide; CYP17A1 inhibitors, such as abiraterone; and somatostatin analogs.

Targeted therapeutics are therapeutics that interfere with specific proteins involved in tumorigenesis and proliferation and may be small molecule drugs; proteins, such as therapeutic antibodies; peptides and peptide derivatives; or protein-small molecule hybrids, such as ADCs. Examples of targeted small molecule drugs include mTor inhibitors, such as everolimus, temsirolimus and rapamycin; kinase inhibitors, such as imatinib, dasatinib and nilotinib; VEGF inhibitors, such as sorafenib and regorafenib; EGFR/HER2 inhibitors, such as gefitinib, lapatinib, and erlotinib; and CDK4/6 inhibitors, such as palbociclib, ribociclib and abemaciclib. Examples of peptide or peptide derivative targeted therapeutics include proteasome inhibitors, such as bortezomib and carfilzomib.

Suitable anti-inflammatory drugs include D-penicillamine, azathioprine and 6-mercaptopurine, cyclosporine, anti-TNF biologicals (e.g., infliximab, etanercept, adalimumab, golimumab, certolizumab, or certolizumab pegol), leflunomide, abatacept, tocilizumab, anakinra, ustekinumab, rituximab, daratumumab, ofatumumab, obinutuzumab, secukinumab, apremilast, acitretin, and JAK inhibitors (e.g., tofacitinib, baricitinib, or upadacitinib).

Immunotherapeutic agents include agents that induce, enhance or suppress an immune response, such as cytokines (IL-2 and IFN-α); immuno modulatory imide drugs, e.g., thalidomide, lenalidomide, pomalidomide, or imiquimod; therapeutic cancer vaccines, e.g., talimogene laherparepvec; cell based immunotherapeutic agents, e.g., dendritic cell vaccines, adoptive T-cells, or chimeric antigen receptor-modified T-cells; and therapeutic antibodies that can trigger antibody-dependent cell-mediated cytotoxicity (ADCC), antibody-dependent cellular phagocytosis (ADCP) or complement-dependent cytotoxicity (CDC) via their Fc region when binding to membrane bound ligands on a cell.

In one embodiment, the present invention relates to the use of a sequentially or concomitantly administered combination of an anti-c-Met antibody or antigen-binding fragment thereof, an anti-c-Met ADC or a pharmaceutical composition as described hereinabove with a therapeutic antibody, a chemotherapeutic agent, and/or an ADC against a cancer-related target other than the c-Met antigen for the treatment of a human solid tumor or hematological malignancy as described hereinabove.

A therapeutically effective amount of the anti-c-Met antibody or antigen-binding fragment thereof, or ADC in accordance with the present invention lies in the range of about 0.01 to about 15 mg/kg body weight, particularly in the range of about 0.1 to about 10 mg/kg body weight, more particularly in the range of about 0.3 to about 10 mg/kg body weight. This latter range corresponds roughly to a flat dose in the range of 20 to 800 mg of the antibody or ADC. The compound of the present invention may be administered weekly, bi-weekly, three-weekly, monthly or six-weekly. Suitable treatment regimens are depending upon the severity of the disease, the age of the patient, the compound being administered, and such other factors as would be considered by the treating physician.

In the context of the invention, treatment is preferably preventing, reverting, curing, ameliorating, and/or delaying the cancer. This may mean that the severity of at least one symptom of the cancer has been reduced, and/or at least a parameter associated with the cancer has been improved.

### General Definitions

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

The word “about” or “approximately” when used in association with a numerical value (e.g., about 10) preferably means that the value may be the given value more or less 1% of the value.

All patent and literature references cited in the present specification are hereby incorporated by reference in their entirety.

The following Examples are offered for illustrative purposes only, and are not intended to limit the scope of the present invention in any way.

### Examples

### Hydrophobic Interaction Chromatography (HIC) for Characterization of ADCs

For analytical HIC, 5-10 μL of sample (1 mg/mL) was injected onto a TSKgel Butyl-NPR column (4.6 mm ID×3.5 cm L, Tosoh Bioscience, Cat. no. 14947). The elution method consisted of a linear gradient from 100% Buffer A (25 mM sodium phosphate, 1.5 M ammonium sulphate, pH 6.95) to 100% of Buffer B (25 mM sodium phosphate, pH 6.95, 20% isopropanol) at 0.4 mL/min over 20 min. A Waters Acquity H-Class UPLC system equipped with PDA-detector and Empower software was used. Absorbance was measured at 214 nm and the retention time of ADCs was determined.

### Size Exclusion Chromatography (SEC) for Characterization of ADCs

For analytical SEC, 5 μL of sample (1 mg/mL) was injected onto a TSKgel G3000SWXL column (5 μm, 7.8 mm ID×30 cm L, Tosoh Bioscience, Cat. no. 08541) equipped with a TSKgel SWXL Guard column (7 μm, 6.0 mm ID×4.0 cm L, Tosoh Bioscience, Cat. no. 08543). The elution method consisted of elution with 100% 50 mM sodium phosphate, 300 mM NaCl, pH 7.5 at 0.6 mL/min for 30 min. The column temperature was maintained at 25° C. A Waters Acquity H-Class UPLC system equipped with PDA-detector and Empower software was used. Absorbance was measured at 214 nm to quantify the amount of HMW species.

### Immunization Protocol and Selection

Mice were repeatedly immunized with recombinant human HGFR/c-Met ECD-Fc fusion protein. B-cells were harvested from the spleen and used to generate 57 hybridomas. These hybridomas were made by polyethylene glycol (PEG)-mediated cell fusion using B cells and murine myeloma cells (accession CVCL_J288) and applying a selection process using HAT medium (hypoxanthine-aminopterin-thymidine medium). Supernatants from the immortalized antibody-secreting hybridoma cell cultures were analyzed for IgG production, antibody isotype and specific binding to HGFR/c-Met using a Luminex bead assay with immobilized c-Met-Fc.

### Screening for Functional Mouse Antibodies

The functionality of the antibodies was determined by analyzing the stimulation of HepG2 cells with Hepatocyte Growth Factor (HGF) in the absence or presence of the antibodies. In an immunoblot analysis, antibodies could be identified that antagonised HGF-induced c-Met and Protein Kinase B (PKB) phosphorylation and were positive in HepG2 flow cytometry. Based on their neutral or antagonistic properties, only eleven of the initially 57 hybridomas were selected and sequenced. One agonistic hybridoma was selected and sequenced to serve as a positive control. Some of the amino acid sequences were mutated to more germline-like sequences to avoid uncommon, potentially instable and low expressing antibody chain sequences. Prior to codon-optimization, one or more amino acid residues in the flanking regions of the VL domain were replaced with amino acid residues from known germline sequences from the international ImMunoGeneTics (IMGT) database (Scaviner et al., Exp. Clin. Immunogenetics 1999, 16.4, 234-240). This process of reverse mutation to germline sequences, also called germlining, was applied to some of the VL domain sequences which were part of framework 1 or the J segment, mouse IGKV-FR1 or IGKJ, respectively. Eleven heavy chain variable domains (VH) and thirteen light chain variable domains (VL) were obtained.

Based on the amino acid sequences, the corresponding DNA coding sequences were codon-optimized for expression in human cells, synthesized and fused to DNA sequences encoding the human antibody constant parts of the IgG1 subclass (HC SEQ ID NO:22, LC SEQ ID NO:23). Batches of 14 chimeric antibodies were made by transgene expression using antibody sequence encoding plasmids expressed in Expi293F cells. Antibodies were purified from the cell supernatant using Protein A affinity purification.

Chimeric antibodies were tested for affinity to full-length cell-surface expressed human and cynomolgus (cyno) c-Met. For this, ExpiCHO-S cells were transiently transfected with plasmid vectors encoding the full length human and cyno c-Met receptors and cultured according the manufacturer's instructions, before being used in antibody binding studies. As a standard plasmid backbone, the commercially available mammalian expression vector pcDNA3.4 (Thermo Fisher Scientific) was used, which contained the full length human or cyno c-Met antigen coding sequence (according to accession number P08581 (SEQ ID NO:24) and A0A2K5UM05 (SEQ ID NO:25), respectively), preceded by a human CMV promoter.

### Humanization

Humanized antibodies were prepared by CDR grafting. The CDRs of two antagonistic and three neutral clones were identified using the CDR-definitions from the numbering system IMGT (Lefranc and Marie-Paule. Immunologist 1999, 7.4, 132-136) and Kabat. Online public databases of human IgG sequences were searched using the mouse VH domain using BLAST search algorithms, and candidate human variable domains were identified. For each variable domain five candidates were selected based on criteria such as framework homology, maintaining key framework residues, canonical loop structure and immunogenicity. The same procedure was repeated for the VL domain of the antibody. All humanized VH variants were combined with all humanized VL variants resulting in 25 humanized variants for each antibody, i.e., 125 in total.

The humanized variants comprising a heavy chain 41C (HC-41C) mutation were synthesized according to the procedure below and their affinity for human and cyno c-Met was measured using ExpiCHO-S cells expressing either human or cyno c-Met.

Transgene Expression of Antibodies and c-Met Antigens
a) Preparation of cDNA Constructs and Expression Vectors

The heavy chain variable domains (VH) of the mouse amino acid sequences were each joined at the N-terminus to a HAVT20 leader sequence (SEQ ID NO:21), and at the C-terminus to the constant domain of a human IgG1 HC according to SEQ ID NO:22. The resulting chimeric amino acid sequences were back-translated into a cDNA sequence and codon-optimized for expression in human cells (Homo sapiens).

Similarly, the chimeric cDNA sequences for the light chain (LC) constructs were obtained by joining the sequences of a suitable secretion signal (also the HAVT20 leader sequence), the light chain variable domains (VL) of the mouse amino acid sequences, and a human IgG K light chain constant region (SEQ ID NO:23), and back-translating the obtained amino acid sequences into a cDNA sequence codon-optimized for expression in human cells (Homo sapiens).

The cDNA sequences encoding the LC and HC of the humanized variants with the HC-41C mutation were obtained using a similar procedure. The HC and LC sequences were joined at the N terminus to the HAVT20 leader sequences (SEQ ID NO:21), and at the C-terminus to the constant domain of the human IgG1 HC according to SEQ ID NO:22 or the human IgG κ light chain constant region (SEQ ID NO:23).

### b) Vector Construction and Cloning Strategy

For expression of the antibody chains and c-Met antigens the commercially available (Thermo Fisher Scientific) mammalian expression vector pcDNA3.4 was used, which contains a CMV:BGHpA expression cassette. The cDNAs for the HC, the LC or the antigen were ligated into the pcDNA3.4 vector, using the restriction sites AscI and NheI. The final vectors containing either the HC, the LC or the c-Met expression cassette (CMV:HC:BGHpA and CMV:LC-BGHpA, respectively) were used for transformation of E. coli NEB 5-alpha cells. Large-scale preparation of the final expression vectors for transfection was performed using Maxi- or Megaprep kits (Qiagen).

### c) Transient Expression of Antibodies in Mammalian Cells

Commercially available Expi293F cells (Thermo Fisher Scientific) were transfected with the expression vectors using the ExpiFectamine transfection agent according to the manufacturer's instructions as follows: 75×10⁷ cells were seeded in 300 mL FortiCHO medium, 300 μg of the expression vector was combined with 800 μL of ExpiFectamine transfection agent and added to the cells. One day after transfection, 1.5 mL Enhancer 1 and 15 mL Enhancer 2 were added to the culture. Six days post transfection, the cell culture supernatant was harvested by centrifugation at 4,000 g for 15 minutes and filtering the clarified harvest over PES bottle filters/MF 75 filters (Nalgene).

d) Transient Expression of c-Met in Mammalian Cells

Commercially available ExpiCHO-S cells (Thermo Fisher Scientific) were transfected with the expression vectors using the ExpiFectamineCHO transfection agent according to the manufacturer's instructions as follows: 1.2×10⁹ cells were seeded in 200 mL ExpiCHO Expression medium, 200 μg of the expression vector was combined with 640 μL of ExpiFectamineCHO transfection agent and added to the cells. One day after transfection, the cell cultures were used for dose-dependent cell binding analysis.

### Cellular Binding Experiments

### Chimeric Antibodies

Cellular binding of the 14 wild-type chimeric and the 14 HC-41C chimeric anti-c-Met antibodies was studied in human c-Met-positive tumor cell lines MKN45, NCI-H596 and PC-3, rhesus c-Met-positive tumor cell line 4 MBr-5, and human c-Met-negative tumor cell line MDA-MB-175-VII. Cells were used around 90% of confluence at the time of an assay, detached with Trypsin-Versene (EDTA) (Lonza) for 5-10 minutes, washed and adjusted to a concentration of 1×10⁶ cells/mL in ice-cold FACS buffer (PBS 1×, 0.1% v/w BSA, 0.02% v/v sodium azide (NaN₃)). Staining was performed in 96-well round-bottomed microtiter plates using ice-cold reagents/solutions at 4° C. to prevent the modulation and internalization of surface antigens. 100,000 cells/well were added to 96-well plates (100 μL/well) and centrifuged at 300×g for 3 minutes. In case of pre-incubation with HGF (paracrine cell lines only), the cells were resuspended in 50 ng/mL recombinant human HGF in FACS buffer (50 μL/well) and incubated for 30 minutes at 4° C., followed by two wash steps with 150 μL FACS buffer. Supernatant was discarded and cells were stained for 30 minutes with 50 μL of each antibody. Serial dilutions were made in ice-cold FACS buffer. Cells were washed twice by centrifugation at 300×g for 3 minutes and resuspended in 50 μL 6000-times diluted secondary F(ab′)₂ goat anti-human IgG (Fc fragment specific) antibody APC-conjugated (Jackson ImmunoResearch). Following a 30-minute incubation on ice, cells were washed again 2 times and resuspended in 150 μL ice-cold FACS buffer for FACS analysis. For analysis of the data, the Median Fluorescence Intensity (MFI) values were obtained.

All chimeric antibodies showed good binding to human c-Met in the cell lines tested. Only five chimeric antibodies also showed good binding to rhesus c-Met in the 4 MBr-5 cell line. As expected there was no difference in binding properties between wild type chimeric antibodies and their HC-41C counterparts. None of the antibodies showed binding in the c-Met-negative cell line, indicating that all antibodies recognize the c-Met receptor specifically. Based upon their high affinity for both human and rhesus c-Met, three wild-type chimeric antibodies and their corresponding HC-41C chimeric antibodies were selected for further development, see Table 1. In the paracrine cell lines, HGF caused a shift in the EC₅₀ values of the six selected chimeric antibodies, indicating that HGF competes with the binding of these antibodies. This effect was not observed for the two non-selected chimeric antibodies and their HC-41C counterparts.

TABLE 1
VH and VL of wild-type (wt) and HC-41C
(41C) chimeric antibodies

| | VH SEQ ID | VL SEQ ID | |
|---|---|---|---|
| Antibody | NO | NO | Functionality |
| wt-chi-mAb1 | 1 | 2 | Partial agonist |
| 41C-chi-mAb1 | 7 | 2 | Partial agonist |
| wt-chi-mAb2 | 3 | 4 | Partial agonist |
| 41C-chi-mAb2 | 8 | 4 | Partial agonist |
| wt-chi-mAb3 | 5 | 6 | Neutral |
| 41C-chi-mAb3 | 9 | 6 | Neutral |

TABLE 2
Characteristics of the three selected hybridomas

| | | K_{D} | | Flow |
|---|---|---|---|---|
| Hybridoma | Subclass | (pM) | Functionality | cytometry |
| Hyb1 | IgG1 | <10 | Antagonist | Positive |
| Hyb2 | IgG1 | <10 | Antagonist | Positive |
| Hyb3 | IgG1 | <10 | Neutral | Positive |

However, although hybridomas Hyb1 and Hyb2 were originally tested (in vitro) and classified as antagonists (Table 2), the corresponding chimeric antibodies wt/41C-chi-mAb1 and wt/41C-chi-mAb2 were evaluated in vitro (in a stimulation of proliferation experiment in NCI-H596 cells) to be partial agonists. From this perspective, although all three wt/41C chimeric mAbs were humanized and studied in further experiments, wt/41C-chi-mAb3 is the most preferred chimeric anti-c-Met antibody to further develop into an ADC for treatment of a cancer indication.

### Humanized Antibodies

Cellular binding of the in total 75 humanized anti-c-Met antibodies (originating from the three selected chimeric antibodies) was studied in human c-Met-positive tumor cell lines MKN45, NCI-H596 and PC-3, rhesus c-Met-positive tumor cell line 4 MBr-5, and human c-Met-negative tumor cell line MDA-MB-175-VII. Cells were used around 90% of confluence at the time of an assay, detached with Trypsin-Versene (EDTA) (Lonza) for 5-10 minutes, washed and adjusted to a concentration of 1×10⁶ cells/mL in ice-cold FACS buffer (PBS 1×, 0.1% v/w BSA, 0.02% v/v sodium azide (NaN₃)). Staining was performed in 96-well round-bottomed microtiter plates using ice-cold reagents/solutions at 4° C. to prevent the modulation and internalization of surface antigens. 100,000 cells/well were added to 96-well plates (100 μL/well) and centrifuged at 300×g for 3 minutes. In case of pre-incubation with HGF, the cells were resuspended in 50 ng/mL recombinant human HGF in FACS buffer (50 μL/well) and incubated for 30 minutes at 4° C., followed by two wash steps with 150 μL FACS buffer. Supernatant was discarded and cells were stained for 30 minutes with 50 μL of each antibody. Serial dilutions were made in ice-cold FACS buffer. Cells were washed twice by centrifugation at 300×g for 3 minutes and resuspended in 50 μL 6000-times diluted secondary F(ab′)₂ goat anti-human IgG (Fc fragment specific) antibody APC-conjugated (Jackson ImmunoResearch). Following a 30-minute incubation on ice, cells were washed again 2 times and resuspended in 150 μL ice-cold FACS buffer for FACS analysis. For analysis of the data, the Median Fluorescence Intensity (MFI) values were obtained.

None of the humanized antibodies showed binding in the c-Met-negative cell line. All humanized antibodies originating from wt/41C-chi-mAb2 and wt/41C-chi-mAb3 showed good binding to both human and rhesus c-Met in the cell lines tested, whereas only five of the 25 humanized antibodies originating from wt/41C-chi-mAb1 showed good binding. Despite the good binding properties of the humanized antibodies originating from wt/41C-chi-mAb2, the hydrophobicity of 20 of the 25 humanized antibodies was dramatically increased, as determined by HIC. This increase in hydrophobicity is an unwanted effect, as the more hydrophobic biological compounds, including antibodies, are, the more quickly they are cleared from circulation (in animal models). A similar observation was made for 14 of the 25 humanized antibodies originating from wt/41C-chi-mAb1. Humanization of wt/41C-chi-mAb3 did not seem to affect binding properties or hydrophobicity.

Table 4 hereinbelow shows the cellular binding EC₅₀ values for the nine HC-41C humanized antibodies (Table 3) that were selected for further development. As shown in Table 4 in the paracrine cell lines, and in accordance with the results for the chimeric antibodies, HGF caused a shift in the EC₅₀ values of the humanized antibodies, indicating that HGF competes with the binding of these antibodies.

Cellular binding of humanized mAb1a (originating from chimeric antibody wt/41C-chi-mAb1) measured on human c-Met antigen-expressing MKN45, NCI-H596 and PC-3 cells, and rhesus c-Met antigen-expressing 4 MBr-5 cells (EC₅₀) was, respectively, 0.16 μg/mL (95% CI: 0.12-0.21 μg/mL), 0.04 μg/mL (95% CI: 0.006-0.225 μg/mL), 0.08 μg/mL (95% CI: 0.02-0.25 μg/mL), and 0.07 μg/mL (95% CI: 0.05-0.09 μg/mL). HGF binding caused a 1.1- to 2.5-fold shift in cellular binding in the paracrine cell lines.

Cellular binding of humanized mAb2a, mAb2b and mAb2c (originating from chimeric antibody wt/41C-chi-mAb2) measured on human c-Met antigen-expressing MKN45, NCI-H596 and PC-3 cells, and rhesus c-Met antigen-expressing 4 MBr-5 cells (EC₅₀) ranged, respectively, from 0.09-0.14 μg/mL, 0.02-0.04 μg/mL, 0.03-0.05 μg/mL, and 0.06-0.09 μg/mL. HGF binding caused a 1.2- to 4.0-fold shift in cellular binding in the paracrine cell lines.

Cellular binding of humanized mAb3a, mAb3b, mAb3c, mAb3d and mAb3e (originating from chimeric antibody wt/41C-chi-mAb3) measured on human c-Met antigen-expressing MKN45, NCI-H596 and PC-3 cells, and rhesus c-Met antigen-expressing 4 MBr-5 cells (EC₅₀) ranged, respectively, from 0.05-0.07 μg/mL, 0.02-0.03 μg/mL, 0.03-0.03 μg/mL, and 0.02-0.04 μg/mL. HGF binding caused a 0.8- to 7.5-fold shift in cellular binding in the paracrine cell lines.

TABLE 3
VH and VL of HC-41C humanized antibodies

| | VH SEQ | VL SEQ |
|---|---|---|
| Antibody | ID NO | ID NO |
| mAb1a | 10 | 11 |
| mAb2a | 12 | 15 |
| mAb2b | 13 | 14 |
| mAb2c | 13 | 15 |
| mAb3a | 16 | 19 |
| mAb3b | 16 | 20 |
| mAb3c | 16 | 18 |
| mAb3d | 17 | 19 |
| mAb3e | 17 | 18 |

TABLE 4
Binding affinities of humanized HC-41C chimeric antibodies
to human and rhesus c-Met ECD

| | | | | |
|---|---|---|---|---|
| EC₅₀ | 95% CI | EC₅₀ | 95% CI | Fold shift |
| (μg/mL) | (μg/mL)* | (μg/mL) | (μg/mL)* | (HGF) |

| | | |
|---|---|---|
| Antibody | without HGF | with 50 ng/mL HGF |

MKN45 (constitutively active cell line)

| | | |
|---|---|---|
| mAb1a | 0.16 | 0.12 to 0.21 |
| mAb2a | 0.09 | 0.07 to 0.13 |
| mAb2b | 0.14 | 0.11 to 0.18 |
| mAb2c | 0.11 | 0.08 to 0.13 |
| mAb3a | 0.07 | 0.06 to 0.10 |
| mAb3b | 0.06 | 0.05 to 0.09 |
| mAb3c | 0.05 | 0.04 to 0.07 |
| mAb3d | 0.07 | 0.05 to 0.10 |
| mAb3e | 0.07 | 0.05 to 0.10 |

NCI-H596 (paracrine cell line)

| | | | | | |
|---|---|---|---|---|---|
| mAb1a | 0.04 | 0.006 to 0.225 | 0.10 | 0.08 to 0.12 | 2.5 |
| mAb2a | 0.02 | 0.004 to 0.113 | 0.08 | 0.07 to 0.11 | 4.0 |
| mAb2b | 0.04 | 0.008 to 0.204 | 0.11 | 0.09 to 0.13 | 2.8 |
| mAb2c | 0.03 | 0.006 to 0.171 | 0.10 | 0.08 to 0.12 | 3.3 |
| mAb3a | 0.02 | 0.003 to 0.101 | 0.08 | 0.05 to 0.11 | 4.0 |
| mAb3b | 0.02 | 0.003 to 0.108 | 0.07 | 0.04 to 0.1 | 3.5 |
| mAb3c | 0.02 | 0.004 to 0.081 | 0.11 | 0.08 to 0.15 | 5.5 |
| mAb3d | 0.02 | 0.004 to 0.128 | 0.15 | 0.11 to 0.21 | 7.5 |
| mAb3e | 0.02 | 0.003 to 0.153 | 0.14 | 0.1 to 0.19 | 7.0 |

PC-3 (paracrine cell line)

| | | | | | |
|---|---|---|---|---|---|
| mAb1a | 0.08 | 0.02 to 0.25 | 0.15 | 0.13 to 0.18 | 1.9 |
| mAb2a | 0.04 | 0.01 to 0.17 | 0.09 | 0.08 to 0.11 | 2.3 |
| mAb2b | 0.05 | 0.01 to 0.21 | 0.13 | 0.1 to 0.16 | 2.6 |
| mAb2c | 0.03 | 0.01 to 0.15 | 0.09 | 0.07 to 0.12 | 3.1 |
| mAb3a | 0.03 | 0.01 to 0.10 | 0.07 | 0.06 to 0.09 | 2.4 |
| mAb3b | 0.03 | 0.01 to 0.11 | 0.06 | 0.04 to 0.09 | 2.0 |
| mAb3c | 0.03 | 0.01 to 0.10 | 0.06 | 0.04 to 0.08 | 1.9 |
| mAb3d | 0.03 | 0.01 to 0.12 | 0.08 | 0.05 to 0.12 | 2.7 |
| mAb3e | 0.03 | 0.01 to 0.14 | 0.09 | 0.05 to 0.14 | 2.8 |

4MBr-5 (paracrine cell line)

| | | | | | |
|---|---|---|---|---|---|
| mAb1a | 0.07 | 0.05 to 0.09 | 0.08 | 0.07 to 0.09 | 1.1 |
| mAb2a | 0.06 | 0.05 to 0.09 | 0.09 | 0.08 to 0.1 | 1.4 |
| mAb2b | 0.09 | 0.06 to 0.14 | 0.15 | 0.14 to 0.17 | 1.7 |
| mAb2c | 0.07 | 0.05 to 0.1 | 0.09 | 0.07 to 0.1 | 1.2 |
| mAb3a | 0.04 | 0.02 to 0.06 | 0.03 | 0.03 to 0.04 | 0.8 |
| mAb3b | 0.02 | 0.01 to 0.04 | 0.04 | 0.03 to 0.04 | 1.8 |
| mAb3c | 0.02 | 0.01 to 0.04 | 0.03 | 0.03 to 0.04 | 1.7 |
| mAb3d | 0.03 | 0.02 to 0.06 | 0.05 | 0.04 to 0.06 | 1.6 |
| mAb3e | 0.03 | 0.02 to 0.05 | 0.04 | 0.03 to 0.05 | 1.4 |

*N = 2

The observed binding affinities (K_{D}-obs) for the unconjugated antibody mAb3b to human and cynomolgus c-Met extracellular domain (ECD) are shown in Table 5. The low K_{D}-obs (0.01 nM) indicates a high affinity between either human or cyno c-Met ECD and the antibody.

Binding analysis was performed on a Surface Plasmon Resonance instrument (Biacore© T200 system, GE Life Sciences) at 37° C. Biotinylated human or cyno c-Met was captured on the surface of a CAPchip made suitable for capture of biotinylated molecules (Sensor Chip CAP, GE Life Sciences) by injection of Biotin Capture reagent for 300 seconds at 2 μL/min on flow cell 1 and 2. A dilution of the biotinylated c-Met antigen in running buffer (10 mM HEPES buffer at 25° C., pH 7.4 with 150 mM NaCl, 3 mM EDTA and 0.005% v/v polyoxyethylene (20) sorbitan monolaurate (Surfactant P20) was injected at variable contact times to obtain different capture levels at 5 μL/min. The dilution and contact time for c-Met variant was estimated with the aim for a capture level around 20 RU. After a one-minute baseline, the mAb3b sample was injected in five increasing concentrations (0.037, 0.11, 0.33, 1 and 3 nM) at 30 μL/mL for 60 seconds with a 900-second dissociation time. The Rₘₐₓ for the interactions was between 5-10 RU. Regeneration was performed with 6 M guanidine-HCl, 0.25 M NaOH solution (120 seconds with flow rate of 30 L/min). Double blank subtraction was performed on the obtained sensorgrams subtracting the signal of a blank reference flow channel and a running buffer injection. Sensorgrams were made using Biacore© T200 evaluation software (v3.1). The sensorgrams were fitted to a 1:1 Langmuir binding model and subsequently evaluated for the goodness of the fit (chi2), the uniqueness of the fit (U-value) and by visual inspection and biological relevance.

TABLE 5
Binding affinities of mAb3b to human
and cynomolgus c-Met ECD

| | mAb3b |
|---|---|
| Receptor | KD-obs (nM) |
| Human c-Met ECD | <0.01 |
| Cyno c-Met ECD | <0.01 |

### Internalization Studies

Internalization of mAb3b was studied in human c-Met-positive tumor cell lines MKN45, NCI-H441 and PC-3. Cells (100,000 cells/well of a 96-well round bottomed microtiter plate) were incubated for 30 minutes at 4° C. with 50 μL 10 μg/mL mAb3b or appropriate non-binding control antibody. After a wash step with ice-cold complete growth medium (CGM) consisting of RPMI (Lonza) supplemented with 10% heat inactivated (HI) fetal bovine serum (FBS) (Gibco) and 80 U/mL penicillin/streptomycin (Lonza), the cells were incubated for 30 minutes at 4° C. with 50 μL Alexa Fluor 488 (AF488)-labeled Fab Fragment goat anti-human IgG (1:600 dilution) (Jackson Immunoresearch). After a wash step with ice-cold CGM, cells were resuspended in 150 μL pre-warmed CGM and transferred to polypropylene tubes, one for each time point (0 h, 0.5 h, 1 h, 3 h, 24 h) and placed into a 37° C. water bath to initiate internalization. After the indicated incubation times, cells were washed once with ice-cold FACS buffer consisting of 1×PBS (Lonza) supplemented with 0.1% v/w BSA (Sigma) and 0.02% sodium azide solution (Sigma) to stop the internalization. The remaining surface expression was visualized after quenching with 50 μL of anti-Alexa Fluor 488 rabbit IgG Ab (1:30 diluted in ice-cold FACS buffer) (Molecular Probes, Life Technologies) for 10 minutes at 4° C. Another 100 μL ice-cold FACS buffer was added before measurement. Identical unquenched tubes were determined for the total fluorescence at each time point. Fluorescence intensities were determined by flow cytometry (BD FACSVerse, Franklin Lakes, NJ) and indicated as the Median Fluorescence Intensity (MFI). Internalization was quantified by calculating the percentage of internalization with the formula described.

$$\% Internalization = 1 - ( {N}_{1} - {Q}_{1} ) / {N}_{1} - ( {N}_{1} * {Q}_{0} / {N}_{0} ) * 100 \%$$

N₁=Unquenched MFI at each time point
Q₁=Quenched MFI at each time point
Q₀=Quenched MFI for the time point 0 h
N₀=Unquenched MFI for the time point 0 h

A limitation of this approach is the incomplete quenching of the cell-surface bound AF488-labeled mAb. This is in line with the supplier's instructions that described a maximum quenching of the AF488 dye up to 90% or lower in the case of conjugated AF488 dye.

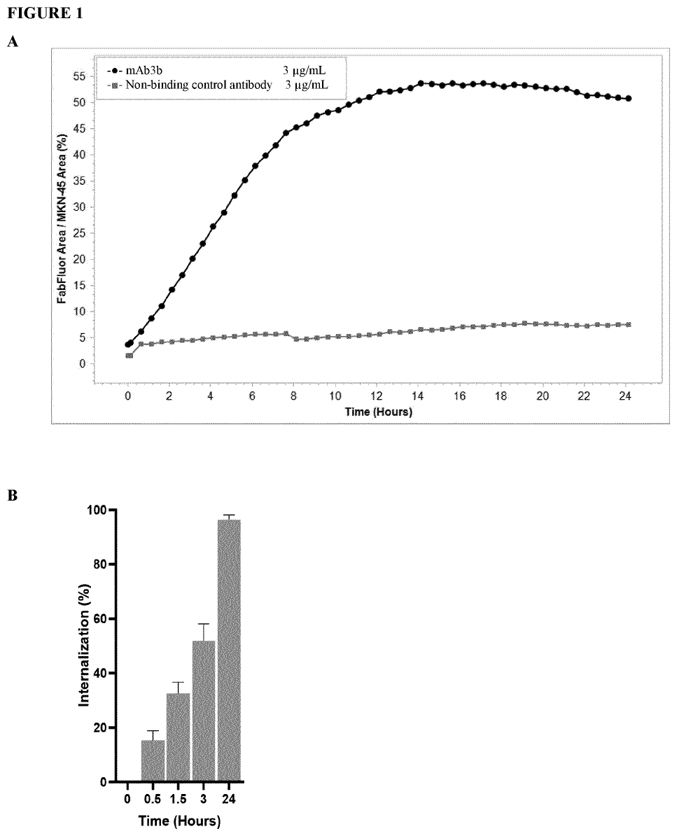

Efficient internalization was shown for mAb3b in each of the cell lines with maximum internalization at 24 hours, as is shown for MKN45 cells in FIG. 1B.

Real time monitoring of internalization of mAb3b was performed in MKN45 cells. Cells (18,750 cells/well) in complete growth medium were plated in 96-well plates (50 μL/well). After overnight incubation at 37° C., 5% CO₂, 3 μg/mL pre-labelled mAb3b with human FabFluor-pH red fluorescent dye (Sartorius) was added to the cells (total volume of 100 μL/well). The real-time live-cell analysis of internalization was assessed by imaging the plates in the IncuCyte S3 instrument, scanning phase and red fluorescence with a 10× objective, 2 images per well, every 30 minutes during 48 hours. The Cell-by-Cell adherent module of the IncuCyte S3 software was used to mask and count the red fluorescence area and the total cell area and plotted these (FabFluor Red Area/MKN45 Area) in a graph versus time (FIG. 1A). The intensity of FabFluor increases during pH-dependent routing to the lysosomes, with the highest fluorescence at pH 4.7.

### Protocols for Site-Specific and Wild-Type Conjugation

### General Conjugation Protocol for Conjugation Via Partially Reduced Endogenous Disulfides (Wild-Type (Wt) Conjugation)

A solution of antibody (5-10 mg/mL in 4.2 mM histidine, 50 mM trehalose, pH 6) was diluted with EDTA (25 mM in water, 4% v/v). The pH was adjusted to ˜7.4 using TRIS (1 M in water, pH 8) after which TCEP (10 mM in water, 1-3 equivalents depending on the antibody and the desired DAR) was added and the resulting mixture was incubated at room temperature (RT) for 1-3 hours. Dimethylacetamide (DMA) was added followed by a solution of linker-drug (10 mM in DMA). The final concentration of DMA was 5-10%. The resulting mixture was incubated at RT in the absence of light for 1-16 hours. In order to remove the excess of linker-drug, activated charcoal was added and the mixture was incubated at RT for 1 hour. The charcoal was removed using a 0.2 m PES filter and the resulting ADC was formulated in 4.2 mM histidine, 50 mM trehalose, pH 6 using a Vivaspin centrifugal concentrator (30 kDa cut-off, PES). Finally, the ADC solution was sterile filtered using a 0.22 μm PES filter.

### General Site-Specific Conjugation Protocol

Site-specific conjugates were synthesised according to the procedure as described in either Protocol A or Protocol B.

### 1) Protocol A

A solution of cysteine-engineered antibody (5-10 mg/mL in 4.2 mM histidine, 50 mM trehalose, pH 6) was diluted with EDTA (25 mM in water, 4% v/v). The pH was adjusted to ˜7.4 using TRIS (1 M in water, pH 8) after which TCEP (10 mM in water, 20 equivalents) was added and the resulting mixture was incubated at RT for 1-3 hours. The excess TCEP was removed by either a PD-10 desalting column or a centrifugal concentrator (Vivaspin filter, 30 kDa cut-off, PES) using 4.2 mM histidine, 50 mM trehalose, pH 6.

The pH of the resulting antibody solution was raised to ˜7.4 using TRIS (1 M in water, pH 8) after which dehydroascorbic acid (10 mM in water, 20 equivalents) was added and the resulting mixture was incubated at RT for 1-2 hours. At RT or 37° C., DMA was added followed by a solution of linker-drug (10 mM in DMA). The final concentration of DMA was 5-10%. The resulting mixture was incubated at RT or 37° C. in the absence of light for 1-16 hours. In order to remove the excess of linker-drug, activated charcoal was added and the mixture was incubated at RT for 1 hour. The charcoal was removed using a 0.2 m PES filter and the resulting ADC was formulated in 4.2 mM histidine, 50 mM trehalose, pH 6 using a Vivaspin centrifugal concentrator (30 kDa cut-off, PES). Finally, the ADC solution was sterile filtered using a 0.22 m PES filter.

### 2) Protocol B

A solution of cysteine-engineered antibody (500 μL, 40 mg/mL in 15 mM histidine, 50 mM sucrose, 0.01% polysorbate-20, pH 6) was diluted with 100 mM histidine, pH 5 (1300 μL). 2-(Diphenylphosphino)benzenesulfonic acid (diPPBS) (426 μL, 10 mM in water, 32 equivalents) was added and the resulting mixture was incubated at RT for 16-24 hours. The excess diPPBS was removed by a centrifugal concentrator (Vivaspin filter, 30 kDa cut-off, PES) using 4.2 mM histidine, 50 mM trehalose, pH 6.

The pH of the resulting antibody solution was raised to ˜7.4 using TRIS (1 M in water, pH 8). At RT or 37° C., DMA was added followed by a solution of linker-drug (10 mM in DMA). The final concentration of DMA was 5-10%. The resulting mixture was incubated at RT or 37° C. in the absence of light for 1-16 hours. In order to remove the excess of linker-drug, activated charcoal was added and the mixture was incubated at RT for 1 hour. The charcoal was removed using a 0.2 m PES filter and the resulting ADC was formulated in 4.2 mM histidine, 50 mM trehalose, pH 6 using a Vivaspin centrifugal concentrator (30 kDa cut-off, PES). Finally, the ADC solution was sterile filtered using a 0.22 m PES filter.

Site-Specific Conjugation Protocol for mAb3b

To a solution of mAb3b (10-12 mg/mL in 100 mM histidine pH 5) diPPBS (10 mM in water, 16-32 equivalents) was added and the resulting mixture was incubated at RT overnight. The excess diPPBS was removed by Vivaspin centrifugal concentrator (30 kDa cut-off, PES) using 4.2 mM histidine, 50 mM trehalose, pH 6. DMA was added followed by a solution of linker-drug (10 mM in DMA). The final concentration of DMA was 10%. The resulting mixture was incubated overnight at RT in the absence of light. In order to remove the excess of linker-drug, activated charcoal was added and the mixture was incubated at RT for 1 hour. The coal was removed using a 0.2 μm PES filter and the resulting ADC was formulated in 4.2 mM histidine, 50 mM trehalose, pH 6 using a Vivaspin centrifugal concentrator (30 kDa cut-off, PES). Finally, the ADC solution was sterile filtered using a 0.22 μm PES filter.

### In Vitro Cytotoxicity of ADCs

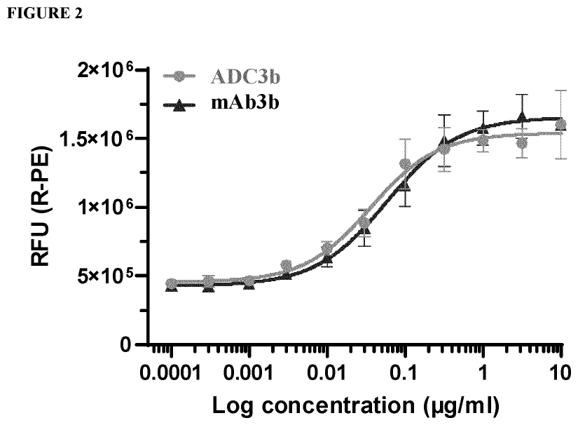

Unconjugated antibodies and ADCs had equal binding affinities on c-Met-expressing MKN45 cells as is shown for antibody mAb3b and antibody-drug conjugate ADC3b in FIG. 2 (mean±S.E.M. of two experiments performed in duplicate). The antigen binding properties of the ADC were thus unaffected by the attached duocarmycin derivative linker-drug.

In vitro cytotoxicity of chimeric and humanized anti-c-Met ADCs (Table 6) was determined in human tumor cell lines with varying levels of c-Met expression. Cells in complete growth medium were plated in 96-well plates (90 or 80 μL/well) and incubated at 37° C., 5% CO₂ at the following cell densities: 2500 MKN-45, 1000 PC-3, 2500 NCI-H596, and 5000 MDA-MB-175-VII cells per well. After an overnight incubation 10 μL of ADC or 10 μL ADC and 10 μL HGF (500 ng/mL) was added. Serial dilutions of the ADC were made in culture medium. Cell viability was assessed after 6 days using the CellTiter-Glom (CTG) luminescent assay kit (Promega Corporation, Madison, WI) according to the manufacturer's instructions. Percentage survival was calculated by dividing the measured luminescence for each ADC concentration with the average mean of untreated cells (only growth medium) multiplied with 100.

Results are shown in Table 7 below. As expected, the non-binding control ADC (rituximab-vc-seco-DUBA) had an effect on the growth of the c-Met-expressing tumor cells only at high concentrations. All anti-c-Met ADCs were inactive (IC₅₀>10 nM) on MDA-MB-175-VII, a c-Met-negative human tumor cell line.

There were no significant differences in the potencies of the HC-41C humanized anti-c-Met ADCs in the various cell lines. There was no effect of HGF (50 ng/mL) on cytotoxicity in PC-3 cells. The efficacy of the NCI-H596 cells stimulated with HGF reached the same level as the NCI-H596 cells without HGF stimulation. Therefore, proliferation was induced in NCI-H596 cells stimulated with 50 ng/mL HGF relative to cells without HGF. HGF did not induce the cytotoxicity.

TABLE 6
HC-41C chimeric and humanized ADCs

| ADC | Antibody | Linker-drug | DAR |
|---|---|---|---|
| ADC1 | 41C-chi-mAb1 | vc-seco-DUBA | 1.5 |
| ADC1a | mAbla | vc-seco-DUBA | 1.7 |
| ADC2 | 41C-chi-mAb2 | vc-seco-DUBA | 1.7 |
| ADC2a | mAb2a | vc-seco-DUBA | 1.7 |
| ADC2b | mAb2b | vc-seco-DUBA | 1.7 |
| ADC2c | mAb2c | vc-seco-DUBA | 1.7 |
| ADC3 | 41C-chi-mAb3 | vc-seco-DUBA | 1.1 |
| ADC3a | mAb3a | vc-seco-DUBA | 1.9 |
| ADC3b | mAb3b | vc-seco-DUBA | 1.9 |
| ADC3c | mAb3c | vc-seco-DUBA | 1.8 |
| ADC3d | mAb3d | vc-seco-DUBA | 1.8 |
| ADC3e | mAb3e | vc-seco-DUBA | 1.9 |

TABLE 7
In vitro cytotoxicity of chimeric and humanized ADCs in human tumor cells expressing c-Met

| | MKN-45 | | PC-3 | | | | NCI-H596 | | | |
|---|---|---|---|---|---|---|---|---|---|---|
| | −HGF | | −HGF | | +HGF | | −HGF | | +HGF | |
| | IC₅₀ | Efficacy | IC₅₀ | Efficacy | IC₅₀ | Efficacy | IC₅₀ | Efficacy | IC₅₀ | Efficacy |
| ADC | (nM) | (%) | (nM) | (%) | (nM) | (%) | (nM) | (%) | (nM) | (%) |
| ADC1 | 0.73 | 72 | 0.069 | 41 | 0.185 | 42 | >10 | 16 | 0.38 | 29 |
| ADC1a | 0.42 | 84 | 0.6¹ | 78 | ~1.22 | 77 | >10 | 14 | >10 | 40 |
| | | | 224² | | | | | | | |
| ADC2 | 0.44 | 76 | 0.05 | 69 | ~10 | 69 | >10 | 6 | >10 | 32 |
| | | | 84 | | | | | | | |
| ADC2a | 0.44 | 86 | 0.06 | 77 | ~0.74 | 76 | >10 | 8 | >10 | 26 |
| | | | 40 | | | | | | | |
| ADC2b | 0.40 | 84 | 0.05 | 77 | ~2.46 | 75 | >10 | 10 | >10 | 34 |
| | | | 100 | | | | | | | |
| ADC2c | 0.41 | 84 | 0.08 | 76 | ~1.97 | 75 | >10 | 3 | >10 | 32 |
| | | | 58 | | | | | | | |
| ADC3 | 0.77 | 50 | 0.076 | 24 | 0.224 | 30 | >10 | 16 | 1.86 | 53 |
| ADC3a | 0.55 | 85 | 0.05 | 73 | ~0.71 | 72 | >10 | 3 | 2.14 | 43 |
| | | | 5.77 | | | | | | | |
| ADC3b | 0.38 | 84 | 0.05 | 75 | ~1.01 | 75 | >10 | 10 | 1.47 | 44 |
| | | | — | | | | | | | |
| ADC3c | 0.41 | 84 | 0.04 | 73 | ~1.85 | 72 | >10 | 10 | 1.89 | 44 |
| | | | 112 | | | | | | | |
| ADC3d | 0.50 | 82 | 0.04 | 74 | ~2.49 | 73 | >10 | 5 | 2.17 | 41 |
| | | | 54 | | | | | | | |
| ADC3e | 0.46 | 81 | 0.04 | 74 | ~2.86 | 74 | >10 | 11 | 2.52 | 44 |
| | | | 541 | | | | | | | |
| Non-binding | >10 | 0 | >10 | 36 | >10 | 36 | >10 | 7 | >10 | 18 |
| control ADC | | | | | | | | | | |
| Free drug | 0.17 | 97 | 0.06 | 97 | 0.07 | 97 | ~4.35 | 81 | 0.18 | 88 |

* MKN-45 has ~150,000 c-Met receptors/cell; PC-3 has ~41,000 c-Met receptors/cell; NCI-H596 has ~35,000 c-Met receptors/cell
¹IC₅₀¹
²IC₅₀²

In a separate experiment, in vitro cytotoxicity of ADC3b was determined in human tumor cell lines with varying levels of c-Met expression. Cells in complete growth medium were plated in 384-well plates (45 μL/well) and incubated at 37° C., 5% CO₂ at the following cell densities: 650 MKN-45, 600 EBC-1, 250 PC-3, 400 KP-4, 2000 NCI-H441, 2500 Hep-G2, 4000 A2780, 1500 HCC-1954 and 600 Jurkat NucLight Red cells per well. After an overnight incubation 5 μL of ADC3b was added. Serial dilutions of the ADC were made in culture medium. Cell viability was assessed after 6 days using the fluorescence-based PrestoBlue® Cell Viability Reagent (Invitrogen, Thermo Fisher scientific, USA) and fluorescence-based CyQUANT® Cell Proliferation Assay (Invitrogen, Thermo Fisher scientific, USA) according to the manufacturer's instructions. Percentage survival was calculated by dividing the measured fluorescence for each ADC concentration with the average mean of untreated cells (only growth medium) multiplied with 100.

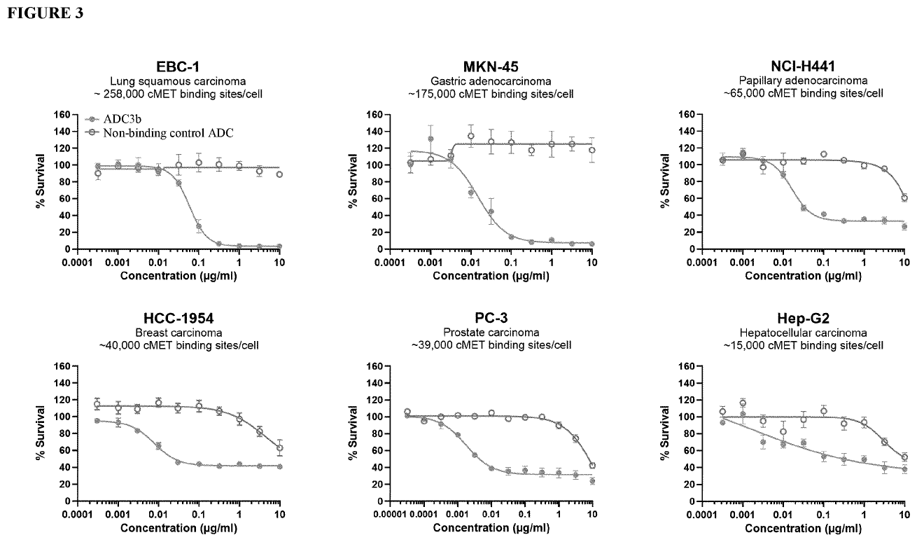

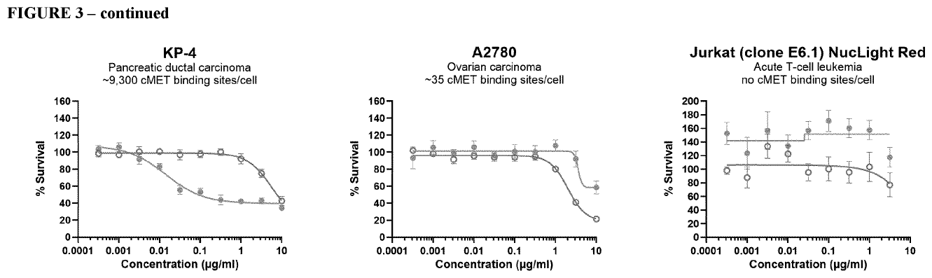

In both read-outs, ADC3b was shown to induce cytotoxicity in human tumor cell lines with high, moderate and low c-Met expression (FIG. 3 shows the read-out of the CyQUANT® Cell Proliferation Assay). Cell survival in FIG. 3 is presented as a mean±S.E.M. of two experiments performed in triplicate. No cytotoxicity was observed for the non-binding control. No cytotoxicity was observed for ADC3b in c-Met negative A2780 (about c-Met antigen binding sites per cell) or Jurkat NucLight Red cells (0 c-Met antigen binding sites per cell).

### Bystander Cytotoxicity of ADCs

Bystander cytotoxicity was determined in c-Met-negative Jurkat NucLight Red cells 1:1 co-cultured with c-Met positive MKN45 cells. 5000 cells/well of each cell type (1:1 ratio) was added in their own culture medium to 96-well plates pre-coated with Poly-L-Omithine (0.1 mg/mL, Sigma). After an overnight incubation at 37° C., 5% CO₂, 10 μL of 1 μg/mL ADC3b or non-binding control ADC (rituximab-vc-seco-DUBA) was added. Live cell analysis of the proliferation of co-cultured c-Met negative Jurkat NucLight Red cells was assessed by imaging the plates in the IncuCyte S3 instrument, scanning phase and fluorescence with a 10× objective, 4 images per well, every 6 hours during 6 days.

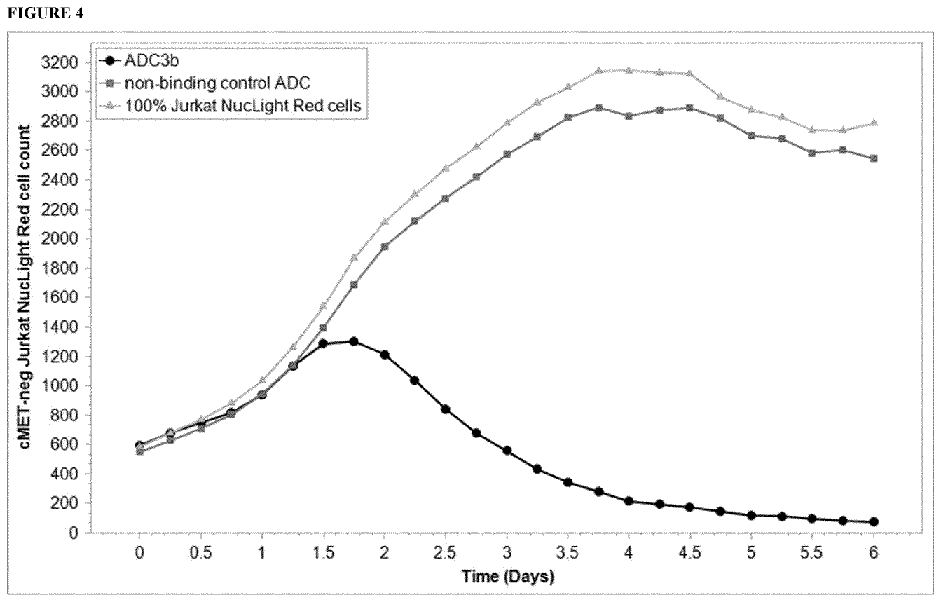

ADC3b is capable of inducing a bystander killing effect in neighbouring cells that do not express c-Met (FIG. 4).

Evaluation of the Chimeric Antibody-Drug Conjugates (ADCs) in a Subcutaneous Xenograft Gastric MKN-45 Tumor Model in Female ces1c KO Mice In Vivo

The in vivo efficacy of the chimeric anti-c-Met ADCs was evaluated in the MKN-45 (gastric adenocarcinoma) cell line xenograft model in B6.Ces1ctm1.1Loc.Foxn1nu mice. These mice lack exon 5 of the Ces1c gene leading to the abolishment of the function of the enzyme. The MET gene is amplified in this cell line; immunohistochemical staining confirmed the high expression of c-Met on the cell surface.

Tumors were induced by subcutaneously injecting 5×10⁶ MKN-45 tumor cells in 200 μL PBS:Matrigel 1:1 into the subcutaneous space of the left flank of all participating mice using a 30 G needle syringe. Animal weights were measured three times weekly. Primary tumors were measured by caliper and tumor volume was calculated according to the formula W²×L/2 (L=length and W=the perpendicular width of the tumor, L>W). After primary tumors had reached a volume of approximately 100 mm³, tumor-bearing animals were randomised over the treatment groups according to tumor volumes and were dosed the same day or the following day with a single injection of 10 mg/kg ADC1 (DAR 1.5), ADC2 (DAR 1.7), or ADC3 (DAR 1.1). The mice were dosed on the same day of grafting or the following day.

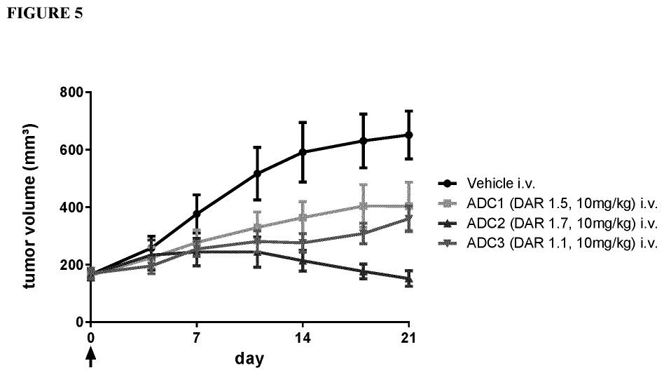

All three chimeric ADCs tested showed significant anti-tumor activity, as shown in FIG. 5.

Evaluation of the Humanized Antibody-Drug Conjugates (ADCs) in a Subcutaneous Xenograft Gastric MKN-45 Tumor Model in Female ces1c KO Mice In Vivo

The in vivo efficacy of the humanized anti-c-Met ADCs was evaluated in the MKN-45 (gastric adenocarcinoma) cell line xenograft model in B6.Ces1ctm1.1Loc.Foxn1nu mice using the same protocol as hereinabove.

After primary tumors have reached a volume of approximately 100 mm³, tumor-bearing animals were randomised over the treatment groups according to tumor volumes and were dosed on the day of randomization or on the following day with a single injection of ADC1a derived from ADC1, ADC2a, ADC2b and ADC2c derived from ADC2, and ADC3a, ADC3b, ADC3c, ADC3d and ADC3e derived from ADC3.

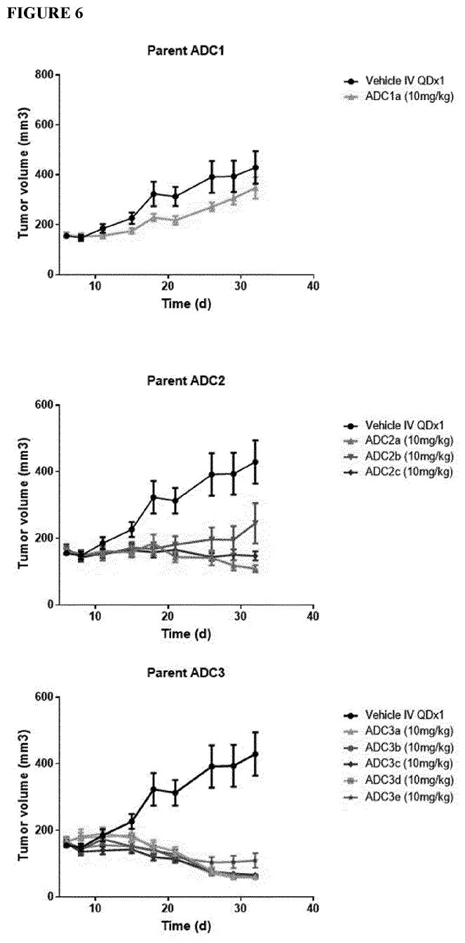

The ADCs based on the humanized antibodies derived from the chimeric mAb used in ADC3 showed the highest anti-tumor activity, as shown in FIG. 6.

### Evaluation of the Antitumoral Efficacy of Humanized Antibody-Drug Conjugates (ADCs) in Patient-Derived Breast Cancer Xenograft Model MAXF574

The in vivo efficacy of the three humanized anti-c-Met ADCs ADC3a, ADC3b, and ADC3c was evaluated in a patient-derived xenograft model of invasive ductal breast carcinoma, MAXF574 in the B6-Ces1ctm1.1Loc.CB17 Prkdc^{scid} mouse strain. These mice lack exon 5 of the Ces1c gene leading to the abolishment of the function of the enzyme. The MET gene is not amplified in this tumor; immunohistochemical staining confirmed moderate to high expression of c-Met on the cell surface.

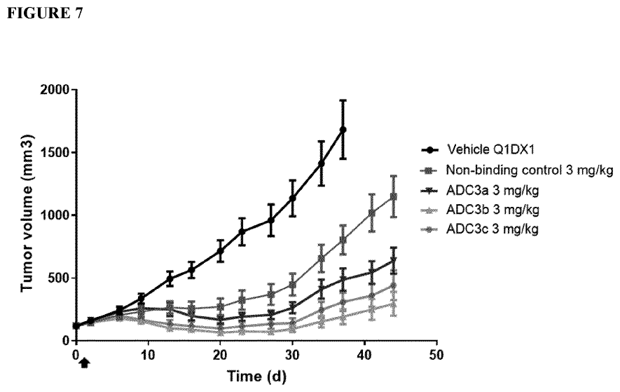

Tumor fragments were obtained from xenografts in serial passage in nude mice. After removal from the donor mice, tumors were cut into fragments (3-4 mm edge length) and placed in PBS containing 10% penicillin/streptomycin. Recipient animals were anesthetized by inhalation of isoflurane and received unilateral tumor implants subcutaneously in the flank. Tumor growth was monitored twice weekly. Tumor volumes were determined by two-dimensional measurement with a digital caliper. Tumor volumes were calculated according to the formula: tumor volume=(1×w²)×0.5, where l=length and w=width (in mm) of the tumor. When tumor implant volumes approached the target range of 80 to 250 mm³ mice were randomized over the treatment groups, aiming at comparable median and mean group tumor volumes. The mice were dosed the same day or the following day with a single injection of 3 mg/kg ADC3a, ADC3b, or ADC3c. A vehicle and non-binding control ADC were included. As shown in FIG. 7, the non-binding control ADC showed some anti-tumor activity, indicative of bystander activity. The three anti-c-Met ADCs showed additional target mediated anti-tumor activity. Of the three ADCs evaluated, ADC3b showed the highest anti-tumor activity.

Dose-Response Studies with ADC3b in Patient-Derived Cancer Xenograft Models LXFL1176 (Lung) and HNXF1905 (Head-and-Neck)

Dose-response studies were performed two patient-derived xenograft models: 1) LXFL1176, a large cell carcinoma derived a lymph node metastasis, and 2) HNXF1905, a primary squamous cell carcinoma from the supraorbital area. The tumor explants were grafted in B6-Ces1ctm1.1Loc.CB17 Prkdc^{scid} mice using the same protocol as described hereinabove. The MET gene is not amplified in either tumor; immunohistochemical staining confirmed high c-Met expression on the cell surface in the LXFL1176 model, and moderate c-Met expression in the HNXF1905 model.

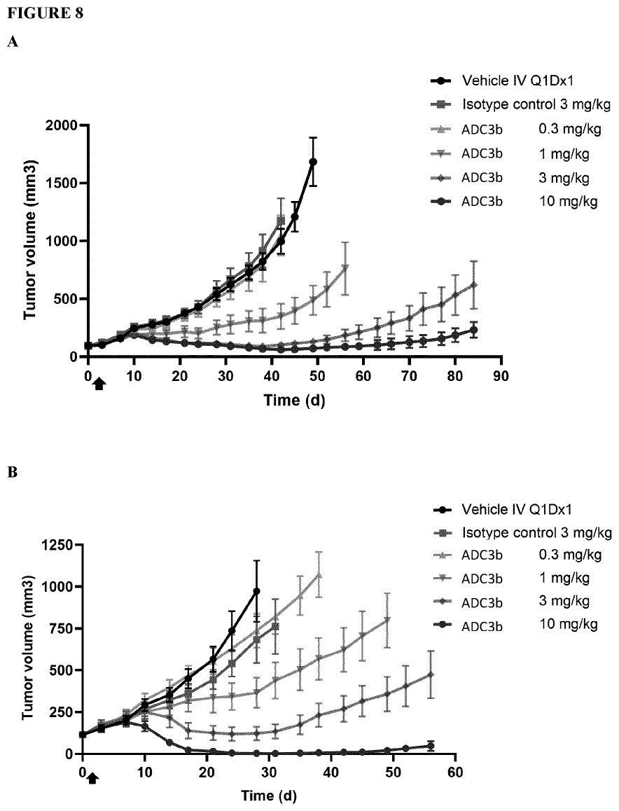

The mice were dosed on the day of randomization or on the following day with a single injection of 0.3, 1, 3 or 10 mg/kg ADC3b. A vehicle and non-binding isotype control ADC were included. The non-binding control ADC showed little to no anti-tumor activity, which indicates that there is little bystander activity in these models. In both models, dose-dependent anti-tumor activity was demonstrated. The efficacy of ADC3b in head and neck cancer PDX model HNXF1905 is shown in FIG. 8A, the efficacy in lung cancer PDX model LXFL1176 in FIG. 8B.

### In Vivo Toxicity Study in Cynomolgus Monkey

The in vivo toxicity of ADC3b was evaluated in male and female cynomolgus monkeys. Monkeys (5 M+5F/group) were dosed with ADC3b (5, 15 or 25 mg/kg Q3W, i.v. infusion).

ADC3b showed a remarkably mild toxicity profile in a 4-cycle pivotal toxicity and PK study in cynomolgus monkey, given c-Met's expression in many different normal tissues. The binding of ADC3b to cynomolgus monkey and human c-Met is comparable (as shown hereinabove). It should be noted that in these normal tissues c-Met is not activated as no c-Met phosphorylation is observed in these tissues. Despite this extensive expression the tolerability of ADC3b is surprising. The highest non-severely toxic dose (HNSTD) for ADC3b is estimated to be 15 mg/kg/Q3 weeks. Given that many of the toxic effects of ADCs are observed in (normal) tissues expressing the target (Masson Hinrichs and Dixit, AAPS J. 2015, 17, 1055-1064), it is surprising that the c-Met targeting ADC ADC3b is so well tolerated.

### In Vivo PKPD Studies

Pharmacokinetic/pharmacodynamic studies were performed with anti-c-Met ADC ADC3b in tumor-bearing CES1c KO SCID mice. These mice lack exon 5 of the Ces1c gene leading to the abolishment of the function of the enzyme. Further pharmacokinetic data were obtained from a toxicology study with ADC3b in cynomolgus monkey.

Mice were dosed with ADC3b (0.3, 1, 3 or 10 mg/kg, intravenous (i.v.) bolus injection) and plasma was collected at 1, 48, 168, 336, 504 hours post dosing. Monkeys were dosed with ADC3b (5, 15 or 25 mg/kg, i.v. infusion) and plasma was collected at 1, 6, 24, 48, 96, 192, 360, 504 hours. An LC-MS/MS based assay was used to quantify total antibody, and ligand binding assays were used to quantify conjugated antibody. The conjugated antibody assay captures ADC species that contain at least one linker drug. The results presented in Tables 8, 9 (mice), and 10 (monkey) show that the ADC is very stable and is cleared slowly with a long half-life.

TABLE 8
Pharmacokinetics of ADC3b in lung LXFL1176
tumor bearing Ces1c KO mice

| Mouse | | | | |
|---|---|---|---|---|
| LXFL1176 | Dose | Approximate | Cₘₐₓ | AUCₗₐₛₜ |
| Xenograft | (mg/kg) | t_{1/2} (hrs) | (μg/mL) | (hrs*μg/mL) |
| Total | 0.3 | NC | 6.24 | 388 |
| antibody | 1 | 95.5 | 22.9 | 1870 |
| | 3 | 192 | 69.8 | 7130 |
| | 10 | NC | 222 | 38500 |
| Conjugated | 0.3 | NC | 4.96 | 355 |
| antibody | 1 | 87.5 | 19.0 | 1670 |
| | 3 | NC | 57.4 | 5920 |
| | 10 | NC | 187 | 33900 |

NC: not calculable

TABLE 9
Pharmacokinetics of ADC3b in head and neck
HNXF1905 tumor bearing Ces1c KO mice

| Mouse | | | | |
|---|---|---|---|---|
| HNXF1905 | Dose | Approximate | Cₘₐₓ | AUCₗₐₛₜ |
| Xenograft | (mg/kg) | t_{1/2} (hrs) | (μg/mL) | (hrs*μg/mL) |
| Total | 0.3 | 207 | 3.86 | 1120 |
| antibody | 1 | NC | 11.4 | 3250 |
| | 3 | NC | 73.2 | 11200 |
| | 10 | NC | 181 | 41500 |
| Conjugated | 0.3 | 181 | 4.85 | 896 |
| antibody | 1 | 221 | 11.7 | 2330 |
| | 3 | NC | 46.7 | 7500 |
| | 10 | NC | 130 | 32300 |

NC: not calculable

TABLE 10
Pharmacokinetics of ADC3b in cynomolgus monkey, single dose

| Healthy | Dose | Approximate | Cₘₐₓ | AUCₗₐₛₜ |
|---|---|---|---|---|
| monkey | (mg/kg) | t_{1/2} (hrs) | (μg/mL) | (hrs*μg/mL) |
| Total | 5 | 82.4 (M)/ | 134 (M)/ | 14600 (M)/ |
| antibody | | 79.1 (F) | 141 (F) | 15000 (F) |
| | 15 | 166 (M)/ | 372 (M)/ | 53800 (M)/ |
| | | 143 (F) | 373 (F) | 47300 (F) |
| | 25 | 159 (M)/ | 586 (M)/ | 84900 (M)/ |
| | | NR (F) | 554 (F) | 84500 (F) |
| Conjugated | 5 | 73.7 (M)/ | 147 (M)/ | 13000 (M)/ |
| antibody | | 73.6 (F) | 155 (F) | 13700 (F) |
| | 15 | 143 (M)/ | 371 (M)/ | 43100 (M)/ |
| | | 125 (F) | 390 (F) | 39200 (F) |
| | 25 | 152 (M)/ | 676 (M)/ | 77300 (M)/ |
| | | 172 (F) | 659 (F) | 78900 (F) |

M: male: F: female.
NR: not reportable

1. An antibody or an antigen-binding fragment thereof that specifically binds to the mesenchymal-epithelial transition factor (c-Met), comprising heavy chain (HC) variable region complementarity determining regions (CDRs) HC CDRs 1-3, wherein
the amino acid sequence of HC CDR1 comprises SEQ ID NO:26;
the amino acid sequence of HC CDR2 comprises SEQ ID NO:27; and
the amino acid sequence of HC CDR3 comprises SEQ ID NO:28;
and light chain (LC) variable region complementarity determining regions (CDRs) LC CDRs 1-3, wherein
the amino acid sequence of LC CDR1 comprises SEQ ID NO:29;
the amino acid sequence of LC CDR2 comprises SEQ ID NO:30; and
the amino acid sequence of LC CDR3 comprises SEQ ID NO:31.

2. The antibody or an antigen-binding fragment thereof according to claim 1, which is a humanized antibody or antigen-binding fragment.

3. The antibody or an antigen-binding fragment thereof according to claim 1, wherein the amino acid sequence of the HC variable region is represented by SEQ ID NO:16 and the amino acid sequence of the LC variable region is represented by SEQ ID NO:20.

4. The antibody according to claim 1, which is an intact IgG1 antibody.

5. The antigen-binding fragment according to claim 1, which is a Fab fragment.

6. An antibody-drug conjugate comprising the antibody or antigen-binding fragment according to claim 1 conjugated to a cytotoxic drug through a linker.

7. The antibody-drug conjugate according to claim 6, wherein the cytotoxic drug is a duocarmycin derivative.

8. The antibody-drug conjugate according to claim 7 of formula (III) 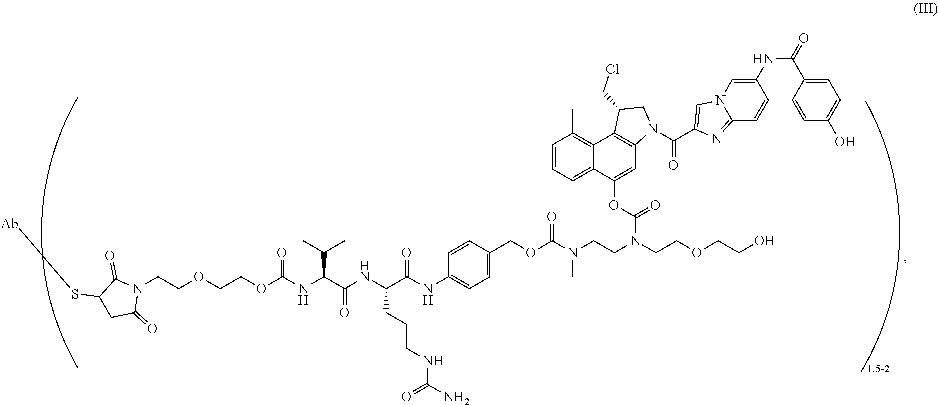
wherein
Ab is an IgG1 antibody;
the HC variable region of Ab is represented by the amino acid sequence of SEQ ID NO:16 and the LC variable region of Ab is represented by the amino acid sequence of SEQ ID NO:20; and
the cytotoxic drug is site-specifically conjugated through the linker to an engineered cysteine on HC position 41 according to Kabat numbering.

9. A pharmaceutical composition comprising an antibody or antigen-binding fragment according to claim 1 and one or more pharmaceutically acceptable excipients.

10. (canceled)

11. A method for the treatment of a c-Met positive human solid tumor or MET-driven hematological malignancy, which comprises administering to a subject in need thereof an effective amount of the antibody or antigen-binding fragment thereof according to claim 1.

12. The method according to claim 11, wherein the c-Met positive human solid tumor is selected from the group consisting of breast cancer; brain cancer; head and neck cancer; thyroid cancer; salivary gland cancer; soft tissue sarcoma; ocular cancer; esophageal cancer; gastric cancer; small intestine cancer; colorectal cancer; urothelial cell cancer; ovarian cancer; uterine cancer; endometrial cancer; cervical cancer; lung cancer; melanoma; liver cancer; pancreatic cancer; non-melanoma skin cancer; prostate cancer; germ cell cancer; and cancer of unknown primary.

13. The method according to claim 11, wherein the MET-driven hematological malignancy is a lymphoid malignancy.

14. The method according to claim 11, which further comprises administering therapeutic antibody, a chemotherapeutic agent and/or an antibody-drug conjugate against a cancer-related target other than the c-Met antigen for use in the treatment of the c-Met positive human solid tumor.

15. The method according to claim 11, wherein said antibody has a cytotoxic drug conjugated thereto through a linker.

16. The method according to claim 12, wherein the lung cancer is selected from non-small cell lung cancer and small-cell lung cancer.

17. The method according to claim 13, wherein the lymphoid malignancy is a mature T and NK neoplasm.

18. The antibody-drug conjugate according to claim 6, wherein said linker is a cleavable linker.

19. A pharmaceutical composition comprising an antibody-drug conjugate according to claim 6 and one or more pharmaceutically acceptable excipients.

20. The pharmaceutical composition according to claim 19, wherein said composition is in the form of a lyophilized powder.

21. The pharmaceutical composition according to claim 9, wherein said composition is in the form of a lyophilized powder.
